(19) United States
(12) Patent Application Publication
(10) Pub. No.: US 2017/0367833 A1
(43) Pub. Date: Dec. 28, 2017

(54) CRUCIATE RETAINING KNEE IMPLANTS AND METHODS FOR IMPLANTING CRUCIATE RETAINING KNEE IMPLANTS

(71) Applicant: ORTHOPEDIC TECHNOLOGY GROUP, Cambridge, MA (US)

(72) Inventor: Kartik Mangudi Varadarajan, Acton, MA (US)

(21) Appl. No.: 15/535,985

(22) PCT Filed: Dec. 15, 2015
(86) PCT No.: PCT/US15/65688

§ 371 Date: Jun. 14, 2017

Related U.S. Application Data
(60) Provisional application No. 62/091,974, filed on Dec. 15, 2014.

(51) Int. Cl. A61F 2/38; A61F 2/46; A61F 2/30

(52) U.S. Cl. CPC A61F 2/3859; A61F 2/389; A61F 2/461; A61F 2002/30553; A61F 2/30942; A61F 2/4684; A61F 2002/30688; A61F 2002/3895; A61F 2002/30112

(57) ABSTRACT

The present invention relates to cruciate ligament retaining knee implants, and instruments and methods for implanting cruciate ligament retaining knee implants.

### CROSS-REFERENCES TO RELATED APPLICATIONS

This application claims priority from U.S. Patent Application No. 62/091,974 filed Dec. 15, 2014.

### STATEMENT REGARDING FEDERALLY SPONSORED RESEARCH

Not Applicable.

### FIELD OF INVENTION

The present invention relates to cruciate ligament retaining knee implants, and instruments and methods for implanting cruciate ligament retaining knee implants.

### BACKGROUND OF INVENTION

Implants used for knee replacement surgery generally comprise one or more femoral components 111, 121, 131 and one or more tibial components (see FIGS. 1A-1C). A tibial component in turn may be composed of tibial baseplate/s and tibial insert/s affixed to the tibial baseplate/s. In knee replacement surgery aiming to retain the anterior cruciate ligament (ACL), the tibial component can take the form of a unicompartmental (Uni—FIG. 1A) implant 110 having a tibial baseplate 112 and a tibial insert 113 that replace one compartment of the native tibia (e.g. medial or lateral); a bi-unicompartmental (Bi-Uni—FIG. 1B) implant 120 composed of two Uni implants 122, 124 having tibial baseplates 127,129 and tibial inserts 126, 128 that replace both medial and lateral compartments of the native tibia; or a Bi-Cruciate (BCR—FIG. 1C) implant 130 composed of a tibial component 132 that replaces both compartments of the native tibia. The BCR tibial component 132 is generally composed of a single-piece tibial baseplate with an anterior-bridge 134 connecting the medial compartment 135 and the lateral compartment 136 of the tibial baseplate, and two tibial inserts 137, 138 affixed to the medial and lateral compartments of the tibial baseplate. In some BCR implants, the tibial insert may also be single-piece, with an anterior-bridge connecting the respective medial and lateral compartments.

The preservation of the ACL allows these implants to better restore the normal motion patterns (kinematics) of the knee following surgery, compared to ACL sacrificing implants. Nonetheless, kinematics of the native knee are not fully restored with these implants. When both medial and lateral compartments of the native tibia are to be replaced, a Bi-Uni or BCR tibial implant component may be used. The advantages of a Bi-Uni tibial implant relative to a BCR tibial implant is that is allows greater flexibility in positioning the medial/lateral tibial components according to the native anatomy of the medial and lateral compartments of the native tibia. However, this flexibility raises the challenge of accurately placing the medial and lateral tibial components relative to each other. The advantage of a BCR implant is that, since a single-piece tibial component (insert and/or baseplate) is used, the relative position of the medial and lateral compartments is maintained and joint loads can be shared between the medial and lateral tibial components. However, the use of a single-piece component requires removal of bone from the anterior region of the tibial eminence to accommodate the anterior-bridge of the BCR tibial implant (FIG. 1C). Further, the ability to match the native anatomy of the medial and lateral compartments of the native tibia is limited.

Accordingly, there remains a need for improved knee implants and instruments to enable accurate placement of knee implants.

### SUMMARY

The present invention relates to cruciate ligament retaining knee implants, instruments and methods for implanting cruciate ligament retaining knee implants.

In one aspect, the invention provides an orthopedic implant having a femoral implant. The femoral implant includes one or both of a medial condyle and a lateral condyle. At least one of the medial condyle and the lateral condyle has a surface region joining a mesial edge and a femur-facing inner surface of the medial condyle or the lateral condyle. The surface region, when viewed in a plane transversely extending from an outer articular surface to the inner surface of the medial condyle or the lateral condyle, has a concave, convex, or chamfered geometry.

In one version of this aspect of the invention, the orthopedic implant is a unicompartmental implant which includes a femoral implant including a medial condyle. A surface region joins a mesial edge and a femur-facing inner surface of the medial condyle. The surface region, when viewed in a plane transversely extending from an outer articular surface to the inner surface of the medial condyle, has a concave, convex, or chamfered geometry.

In one version of this aspect of the invention, the orthopedic implant is a unicompartmental implant that includes a femoral implant having a lateral condyle. A surface region that joins a mesial edge and a femur-facing inner surface of the lateral condyle. The surface region, when viewed in a plane transversely extending from an outer articular surface to the inner surface of the lateral condyle, has a concave, convex, or chamfered geometry.

In one version of this aspect of the invention, the orthopedic implant is a bi-unicompartmental implant configured to replace both medial and lateral compartments of a native tibia. The bi-unicompartmental implant includes the femoral implant which includes a medial condyle and a lateral condyle. A surface region joins a mesial edge and a femur-facing inner surface of the medial condyle. The surface region, when viewed in a plane transversely extending from an outer articular surface to the inner surface of the medial condyle, has a concave, convex, or chamfered geometry.

In one version of this aspect of the invention, the orthopedic implant is a bi-unicompartmental implant configured to replace both medial and lateral compartments of a native tibia. The bi-unicompartmental implant includes a femoral implant which includes a medial condyle and a lateral condyle. A surface region joins a mesial edge and a femur-facing inner surface of the lateral condyle. The surface region, when viewed in a plane transversely extending from an outer articular surface to the inner surface of the lateral condyle, has a concave, convex, or chamfered geometry.

In one version of this aspect of the invention, the orthopedic implant is a bi-unicompartmental implant configured to replace both medial and lateral compartments of a native tibia. The bi-unicompartmental implant includes a femoral implant which includes a medial condyle and a lateral condyle. A surface region joins a mesial edge and a femur-facing inner surface of the medial condyle. The surface region, when viewed in a plane transversely extending from an outer articular surface to the inner surface of the medial condyle, has a concave, convex, or chamfered geometry. A second surface region joins a mesial edge and a femur-facing inner surface of the lateral condyle. The surface region, when viewed in a plane transversely extending from an outer articular surface to the inner surface of the lateral condyle, has a concave, convex, or chamfered geometry.

In one version of this aspect of the invention, the orthopedic implant is a bi-cruciate implant configured to replace both medial and lateral compartments of a native tibia. The bi-cruciate implant includes a femoral implant that includes a medial condyle and a lateral condyle. A surface region joins a mesial edge and a femur-facing inner surface of the medial condyle. The surface region, when viewed in a plane transversely extending from an outer articular surface to the inner surface of the medial condyle, has a concave, convex, or chamfered geometry.

In one version of this aspect of the invention, the orthopedic implant is a bi-cruciate implant configured to replace both medial and lateral compartments of a native tibia. The bi-cruciate implant includes a femoral implant that includes a medial condyle and a lateral condyle. A surface region joins a mesial edge and a femur-facing inner surface of the lateral condyle. The surface region, when viewed in a plane transversely extending from an outer articular surface to the inner surface of the lateral condyle, has a concave, convex, or chamfered geometry.

In one version of this aspect of the invention, the orthopedic implant is a bi-cruciate implant configured to replace both medial and lateral compartments of a native tibia. The bi-cruciate implant includes a femoral implant that includes a medial condyle and a lateral condyle. A surface region joins a mesial edge and a femur-facing inner surface of the medial condyle. The surface region, when viewed in a plane transversely extending from an outer articular surface to the inner surface of the medial condyle, has a concave, convex, or chamfered geometry. A second surface region joins a mesial edge and a femur-facing inner surface of the lateral condyle. The surface region, when viewed in a plane transversely extending from an outer articular surface to the inner surface of the lateral condyle, has a concave, convex, or chamfered geometry.

In one version of this aspect of the invention, the surface region, when viewed in the plane, has a straight line profile.

In one version of this aspect of the invention, the surface region, when viewed in the plane, has a concave curvilinear profile.

In one version of this aspect of the invention, the surface region, when viewed in the plane, has a convex curvilinear profile.

In one version of this aspect of the invention, the surface region, when viewed in the plane, has a concave profile includes a plurality of connected line segments.

In one version of this aspect of the invention, a distance, measured perpendicularly from the inner surface to a normal line to a junction of the surface region and the mesial edge, is in a range of range 0.5 to 7 millimeters.

In one version of this aspect of the invention, a distance, measured perpendicularly from the mesial edge to a normal line to a junction of the surface region and the inner surface, is in a range of range 0.5 to 7 millimeters.

In another aspect, the invention provides an orthopedic implant. The orthopedic implant includes a femoral implant including one or both of a medial condyle and a lateral condyle. At least one of the medial condyle and the lateral condyle is configured such that a posterior thickness and/or a posterodistal condyle thickness of the medial condyle or the lateral condyle is less than a distal condyle thickness of the medial condyle or the lateral condyle.

In one version of this aspect of the invention, the orthopedic implant is a unicompartmental implant that includes a femoral implant including a medial condyle. The medial condyle is configured such that a posterior thickness and/or a posterodistal condyle thickness of the medial condyle is less than a distal condyle thickness of the medial condyle.

In one version of this aspect of the invention, the orthopedic implant is a unicompartmental implant that includes a femoral implant including a lateral condyle. The lateral condyle is configured such that a posterior thickness and/or a posterodistal condyle thickness of the lateral condyle is less than a distal condyle thickness of the lateral condyle.

In one version of this aspect of the invention, the orthopedic implant is a bi-unicompartmental implant configured to replace both medial and lateral compartments of a native tibia. The bi-unicompartmental implant includes a femoral implant including a medial condyle and a lateral condyle. The medial condyle is configured such that a posterior thickness and/or a posterodistal condyle thickness of the medial condyle is less than a distal condyle thickness of the medial condyle.

In one version of this aspect of the invention, the orthopedic implant is a bi-unicompartmental implant configured to replace both medial and lateral compartments of a native tibia. The bi-unicompartmental implant includes a femoral implant including a medial condyle and a lateral condyle. The lateral condyle is configured such that a posterior thickness and/or a posterodistal condyle thickness of the lateral condyle is less than a distal condyle thickness of the lateral condyle.

In one version of this aspect of the invention, the orthopedic implant is a bi-unicompartmental implant configured to replace both medial and lateral compartments of a native tibia. The bi-unicompartmental implant includes a femoral implant including a medial condyle and a lateral condyle. The medial condyle is configured such that a posterior thickness and/or a posterodistal condyle thickness of the medial condyle is less than a distal condyle thickness of the medial condyle. The lateral condyle is configured such that a posterior thickness and/or a posterodistal condyle thickness of the lateral condyle is less than a distal condyle thickness of the lateral condyle.

In one version of this aspect of the invention, the orthopedic implant is a bi-cruciate implant configured to replace both medial and lateral compartments of a native tibia. The bi-cruciate implant includes a femoral implant including a medial condyle and a lateral condyle. The medial condyle is configured such that a posterior thickness and/or a posterodistal condyle thickness of the medial condyle is less than a distal condyle thickness of the medial condyle.

In one version of this aspect of the invention, the orthopedic implant is a bi-cruciate implant configured to replace both medial and lateral compartments of a native tibia. The bi-cruciate implant includes a femoral implant including a medial condyle and a lateral condyle. The lateral condyle is configured such that a posterior thickness and/or a posterodistal condyle thickness of the lateral condyle is less than a distal condyle thickness of the lateral condyle.

In one version of this aspect of the invention, the orthopedic implant is a bi-cruciate implant configured to replace both medial and lateral compartments of a native tibia. The bi-cruciate implant includes a femoral implant including a medial condyle and a lateral condyle. The medial condyle is configured such that a posterior thickness and/or a posterodistal condyle thickness of the medial condyle is less than a distal condyle thickness of the medial condyle. The lateral condyle is configured such that a posterior thickness and/or a posterodistal condyle thickness of the lateral condyle is less than a distal condyle thickness of the lateral condyle.

In one version of this aspect of the invention, a posterior thickness of the medial condyle or the lateral condyle is less than a distal condyle thickness of the medial condyle or the lateral condyle.

In one version of this aspect of the invention, a posterodistal condyle thickness of the medial condyle or the lateral condyle is less than a distal condyle thickness of the medial condyle or the lateral condyle.

In one version of this aspect of the invention, a posterior thickness and a posterodistal condyle thickness of the medial condyle or the lateral condyle are less than a distal condyle thickness of the medial condyle or the lateral condyle.

In another aspect, the invention provides an orthopedic implant. The orthopedic implant includes a femoral implant including one or both of a medial condyle and a lateral condyle. The medial condyle and/or the lateral condyle is configured such that each of a posterior thickness, a posterodistal thickness, and a distal condyle thickness of the medial condyle or the lateral condyle is less than 8 millimeters. The medial condyle and/or the lateral condyle includes a reinforcing structure that extends away from the inner surface of the medial condyle or the lateral condyle. The reinforcing structure is configured to interface with femoral bone.

In one version of this aspect of the invention, the orthopedic implant is a unicompartmental implant that includes a femoral implant having a medial condyle. The medial condyle includes the reinforcing structure.

In one version of this aspect of the invention, the orthopedic implant is a unicompartmental implant that includes a femoral implant having a lateral condyle. The lateral condyle includes the reinforcing structure.

In one version of this aspect of the invention, the orthopedic implant is a bi-unicompartmental implant configured to replace both medial and lateral compartments of a native tibia. The bi-unicompartmental implant includes a femoral implant having a medial condyle and a lateral condyle. The medial condyle includes the reinforcing structure.

In one version of this aspect of the invention, the orthopedic implant is a bi-unicompartmental implant configured to replace both medial and lateral compartments of a native tibia. The bi-unicompartmental implant includes a femoral implant having a medial condyle and a lateral condyle. The lateral condyle includes the reinforcing structure.

In one version of this aspect of the invention, the orthopedic implant is a bi-cruciate implant configured to replace both medial and lateral compartments of a native tibia. The bi-cruciate implant includes a femoral implant having a medial condyle and a lateral condyle. The medial condyle includes the reinforcing structure.

In one version of this aspect of the invention, the orthopedic implant is a bi-cruciate implant configured to replace both medial and lateral compartments of a native tibia. The bi-cruciate implant includes a femoral implant having a medial condyle and a lateral condyle. The lateral condyle includes the reinforcing structure.

In one version of this aspect of the invention, the reinforcing structure comprises a rectangular fin.

In one version of this aspect of the invention, the reinforcing structure comprises a partially cylindrical fin.

In another aspect, the invention provides an orthopedic implant, wherein the orthopedic implant includes a tibial implant including one or both of a medial tibial component and a lateral tibial component. At least one of the medial tibial component and the lateral tibial component is configured such a surface region joining a mesial edge and a tibial-facing surface of the medial tibial component or the lateral tibial component has a rounded profile or a chamfered profile when viewed in a plane coronally extending from an outer edge of the medial tibial component or the lateral tibial component to the mesial edge of the medial tibial component or the lateral tibial component.

In one version of this aspect of the invention, the orthopedic implant is a unicompartmental implant that includes a tibial implant having a medial tibial component. A surface region joins a mesial edge and a tibial-facing surface of the medial tibial component. The surface region has a rounded profile or a chamfered profile when viewed in a plane coronally extending from an outer edge of the medial tibial component to the mesial edge of the medial tibial component.

In one version of this aspect of the invention, the orthopedic implant is a unicompartmental implant that includes a tibial implant having a lateral tibial component. A surface region joins a mesial edge and a tibial-facing surface of the lateral tibial component. The surface region has a rounded profile or a chamfered profile when viewed in a plane coronally extending from an outer edge of the lateral tibial component to the mesial edge of the lateral tibial component.

In one version of this aspect of the invention, the orthopedic implant is a bi-unicompartmental implant configured to replace both medial and lateral compartments of a native tibia. The bi-unicompartmental implant includes a tibial implant having a medial tibial component and a lateral tibial component. A surface region joins a mesial edge and a tibial-facing surface of the medial tibial component. The surface region has a rounded profile or a chamfered profile when viewed in a plane coronally extending from an outer edge of the medial tibial component to the mesial edge of the medial tibial component. The tibial implant includes a second surface region that joins a mesial edge and a tibial-facing surface of the lateral tibial component. The surface region has a rounded profile or a chamfered profile when viewed in a plane coronally extending from an outer edge of the lateral tibial component to the mesial edge of the lateral tibial component.

In one version of this aspect of the invention, the orthopedic implant is a bi-cruciate implant configured to replace both medial and lateral compartments of a native tibia. The bi-cruciate implant includes a tibial implant having a medial tibial component and a lateral tibial component. A surface region joins a mesial edge and a tibial-facing surface of the medial tibial component. The surface region has a rounded profile or a chamfered profile when viewed in a plane coronally extending from an outer edge of the medial tibial component to the mesial edge of the medial tibial component. The tibial implant includes a second surface region that joins a mesial edge and a tibial-facing surface of the lateral tibial component. The second surface region has a rounded profile or a chamfered profile when viewed in a plane coronally extending from an outer edge of the lateral tibial component to the mesial edge of the lateral tibial component.

In one version of this aspect of the invention, the surface region, when viewed in the plane, has a straight line profile.

In one version of this aspect of the invention, the surface region, when viewed in the plane, has a concave curvilinear profile.

In another aspect, the invention provides an orthopedic implant includes a tibial implant including one or both of a medial tibial component and a lateral tibial component. At least one of the medial tibial component and the lateral tibial component has a non-straight mesial edge when viewed in a plane transversely extending from an outer edge of the medial tibial component or the lateral tibial component to the mesial edge of the medial tibial component or the lateral tibial component.

In one version of this aspect of the invention, the orthopedic implant is a unicompartmental implant that includes a tibial implant having a medial tibial component. The medial tibial component has a non-straight mesial edge when viewed in a plane transversely extending from an outer edge of the medial tibial component to the mesial edge of the medial tibial component.

In one version of this aspect of the invention, the orthopedic implant is a unicompartmental implant that includes the tibial implant including the lateral tibial component. The lateral tibial component has a non-straight mesial edge when viewed in a plane transversely extending from an outer edge of the lateral tibial component to the mesial edge of the lateral tibial component.

In one version of this aspect of the invention, the orthopedic implant is a bi-unicompartmental implant configured to replace both medial and lateral compartments of a native tibia. The bi-unicompartmental implant includes a tibial implant having a medial tibial component and a lateral tibial component. The medial tibial component and the lateral tibial component have the non-straight mesial edge.

In one version of this aspect of the invention, the non-straight mesial edge has a concave curvilinear profile.

In one version of this aspect of the invention, the non-straight mesial edge has a concave profile includes a plurality of connected straight line segments.

In one version of this aspect of the invention, the medial tibial component and the lateral tibial component each comprise a tibial baseplate.

In one version of this aspect of the invention, the medial tibial component and the lateral tibial component each comprise a tibial insert.

In another aspect, the invention provides an orthopedic implant including a tibial implant having a medial tibial component and a lateral tibial component. The medial tibial component has a distal surface and a proximal surface. The lateral tibial component has a distal surface and a proximal surface. The proximal surface of the medial compartment has a different posterior slope than the proximal surface of the lateral compartment. In one version of this aspect of the invention, the distal surface of the medial tibial component and the distal surface of the lateral tibial component have the same posterior slope. In another version of this aspect of the invention, the distal surface of the medial compartment has a different posterior slope than the distal surface of the lateral compartment. In another version of this aspect of the invention, the tibial implant is a tibial baseplate that includes the medial tibial component and the lateral tibial component.

In another aspect, the invention provides an orthopedic implant including a tibial implant having one or both of a medial tibial component and a lateral tibial component. At least one of the medial tibial component and the lateral tibial component includes a distal surface having a convex geometry.

In one version of this aspect of the invention, the orthopedic implant is a unicompartmental implant that includes a tibial implant having a medial tibial component. The medial tibial component and the lateral tibial component include a distal surface having a convex geometry.

In one version of this aspect of the invention, the orthopedic implant is a unicompartmental implant that includes a tibial implant having a lateral tibial component. The lateral tibial component includes a distal surface having a convex geometry.

In one version of this aspect of the invention, the orthopedic implant is a bi-unicompartmental implant configured to replace both medial and lateral compartments of a native tibia. The bi-unicompartmental implant includes a tibial implant having a medial tibial component and a lateral tibial component. Each of the medial tibial component and the lateral tibial component includes a distal surface having a convex geometry.

In one version of this aspect of the invention, the orthopedic implant is a bi-cruciate implant configured to replace both medial and lateral compartments of a native tibia. The bi-cruciate implant includes a tibial implant having a medial tibial component and a lateral tibial component. Each of the medial tibial component and the lateral tibial component includes a distal surface having a convex geometry.

In another aspect, the invention provides an orthopedic implant including a tibial implant having a medial tibial component, a lateral tibial component, and an anterior bridge joining the medial tibial component and the lateral tibial component. The anterior bridge is configured such that a superior portion of the anterior bridge drapes over a portion of a tibial eminence. The anterior bridge is configured such that a portion of the anterior-bridge is distal to a distal surface of the medial tibial component and/or a distal surface of the lateral tibial component. In one version of this aspect of the invention, a cavity is formed underneath the anterior bridge. In another version of this aspect of the invention, the tibial implant is configured such that a distal end of the anterior bridge seats in a notch in a tibia when the tibial implant is implanted on the tibia. In another version of this aspect of the invention, the anterior bridge includes a curvilinear outer surface.

In another aspect, the invention provides an orthopedic instrument designed to aid in cutting native tibia bone to accommodate a knee implant. The instrument includes a medial cutting guide and a lateral cutting guide. The instrument is configured to allow relative rotation between the medial cutting guide and lateral cutting guide in at least one of a sagittal, coronal or transverse plane.

In one version of this aspect of the invention, the medial cutting guide includes a medial transverse cutting slot, and the lateral cutting guide includes a lateral transverse cutting slot.

In one version of this aspect of the invention, the medial cutting guide includes a medial tibial eminence cutting slot, and the lateral cutting guide includes a lateral tibial eminence cutting slot.

In one version of this aspect of the invention, one of the medial cutting guide and the lateral cutting guide includes a base section that extends away from a lower section. The other of the medial cutting guide and the lateral cutting guide engages the base section and pivots in a transverse plane with respect to the base section.

In one version of this aspect of the invention, one of the medial cutting guide and the lateral cutting guide engages the other of the medial cutting guide and the lateral cutting guide and pivots in a sagittal plane with respect to the other of the medial cutting guide and the lateral cutting guide.

In one version of this aspect of the invention, one of the medial cutting guide and the lateral cutting guide engages the other of the medial cutting guide and the lateral cutting guide and pivots in a coronal plane with respect to the other of the medial cutting guide and the lateral cutting guide.

In one version of this aspect of the invention, there is provided a saw blade dimensioned to slide within the medial transverse cutting slot, the lateral transverse cutting slot, the medial tibial eminence cutting slot, and the lateral tibial eminence cutting slot.

In another aspect, the invention provides an orthopedic instrument designed to aid in cutting native tibia bone to accommodate a knee implant. The instrument includes a medial cutting guide, a lateral cutting guide, and a base block. The instrument is configured to allow relative rotation between the block base and at least one of the medial cutting guide and the lateral cutting guide in at least one of a sagittal, coronal or transverse plane.

In one version of this aspect of the invention, the instrument is configured to allow relative rotation between the block base and the medial cutting guide in a sagittal plane.

In one version of this aspect of the invention, the instrument is configured to allow relative rotation between the block base and the medial cutting guide in a transverse plane.

In one version of this aspect of the invention, the instrument is configured to allow relative rotation between the block base and the lateral cutting guide in a sagittal plane.

In one version of this aspect of the invention, the instrument is configured to allow relative rotation between the block base and the lateral cutting guide in a transverse plane.

In one version of this aspect of the invention, the medial cutting guide includes a medial transverse cutting slot, and the lateral cutting guide includes a lateral transverse cutting slot.

In one version of this aspect of the invention, the medial cutting guide includes a medial tibial eminence cutting slot, and the lateral cutting guide includes a lateral tibial eminence cutting slot.

In one version of this aspect of the invention, there is provided a saw blade dimensioned to slide within the medial transverse cutting slot, the lateral transverse cutting slot, the medial tibial eminence cutting slot, and the lateral tibial eminence cutting slot.

In another aspect, the invention provides an orthopedic instrument designed to aid in cutting native tibia bone to accommodate a knee implant. The instrument includes a medial cutting guide and a lateral cutting guide. The instrument is configured to allow relative translation between the medial cutting guide and the lateral cutting guide in at least one of a mediolateral, anteroposterior or superoinferior direction.

In one version of this aspect of the invention, the instrument is configured to allow relative translation between the medial cutting guide and the lateral cutting guide in a mediolateral direction.

In one version of this aspect of the invention, the instrument is configured to allow relative translation between the medial cutting guide and the lateral cutting guide in an anteroposterior direction.

In one version of this aspect of the invention, the instrument is configured to allow relative translation between the medial cutting guide and the lateral cutting guide in a superoinferior direction.

In one version of this aspect of the invention, the medial cutting guide includes a medial transverse cutting slot, and the lateral cutting guide includes a lateral transverse cutting slot.

In one version of this aspect of the invention, the medial cutting guide includes a medial tibial eminence cutting slot, and the lateral cutting guide includes a lateral tibial eminence cutting slot.

In one version of this aspect of the invention, there is provided a saw blade dimensioned to slide within the medial transverse cutting slot, the lateral transverse cutting slot, the medial tibial eminence cutting slot, and the lateral tibial eminence cutting slot.

In another aspect, the invention provides an orthopedic instrument designed to aid in cutting native tibia bone to accommodate a knee implant. The instrument includes a medial cutting guide, a lateral cutting guide, and a base block. The instrument is configured to allow relative translation between the block base and at least one of the medial cutting guide and the lateral cutting guide in at least one of a mediolateral, anteroposterior or superoinferior direction.

In one version of this aspect of the invention, the instrument is configured to allow relative translation between the block base and the medial cutting guide in a mediolateral direction.

In one version of this aspect of the invention, the instrument is configured to allow relative translation between the block base and the medial cutting guide in an anteroposterior direction.

In one version of this aspect of the invention, the instrument is configured to allow relative translation between the block base and the medial cutting guide in a superoinferior direction.

In one version of this aspect of the invention, the instrument is configured to allow relative translation between the block base and the lateral cutting guide in a mediolateral direction.

In one version of this aspect of the invention, the instrument is configured to allow relative translation between the block base and the lateral cutting guide in an anteroposterior direction.

In one version of this aspect of the invention, the instrument is configured to allow relative translation between the block base and the lateral cutting guide in a superoinferior direction.

In one version of this aspect of the invention, the medial cutting guide includes a medial transverse cutting slot, and the lateral cutting guide includes a lateral transverse cutting slot.

In one version of this aspect of the invention, the medial cutting guide includes a medial tibial eminence cutting slot. The lateral cutting guide includes a lateral tibial eminence cutting slot.

In one version of this aspect of the invention, there is provided a saw blade dimensioned to slide within the medial transverse cutting slot, the lateral transverse cutting slot, the medial tibial eminence cutting slot, and the lateral tibial eminence cutting slot.

In another aspect, the invention provides an orthopedic instrument designed to aid in cutting native tibia bone to accommodate a knee implant. The instrument includes a cutting guide that is configured to allow varying relative resection depths and/or relative posterior slopes between a medial bone resection and a lateral bone resection.

In one version of this aspect of the invention, the cutting guide includes a medial transverse cutting slot and a lateral transverse cutting slot. The medial transverse cutting slot is a first distance from a base wall of the cutting guide. The lateral transverse cutting slot is a second distance from the base wall of the cutting guide. The first distance and the second distance are different.

In one version of this aspect of the invention, the cutting guide includes a medial transverse cutting slot and a lateral transverse cutting slot. At least one the medial transverse cutting slot and the lateral transverse cutting slot includes an inlet at a first distance from a first end of a base wall of the cutting guide and an outlet at a second distance from a second end the base wall of the cutting guide. The first distance and the second distance are different.

In one version of this aspect of the invention, the cutting guide includes a medial tibial eminence cutting slot and a lateral tibial eminence cutting slot.

In another aspect, the invention provides an orthopedic instrument designed to aid in cutting native tibia bone to accommodate a knee implant. The instrument comprises a cutting guide including a first section having an edge and a second section having a transverse cutting slot. The edge is configured to use a cut of one of a medial tibial surface or a lateral tibial surface as a reference to guide resection of the other of the medial tibial surface or the lateral tibial surface.

In one version of this aspect of the invention, the second section includes a tibial eminence cutting slot.

In one version of this aspect of the invention, the transverse cutting slot includes an inlet at a first distance from a first end of a base wall of the cutting guide and an outlet at a second distance from a second end the base wall of the cutting guide. The first distance and the second distance are different.

In another aspect, the invention provides an orthopedic instrument for use with an implant including a medial tibial component and a separate lateral tibial component. The instrument includes a holder configured to temporarily hold the medial tibial component and the lateral tibial component in a desired relative orientation to each other.

In one version of this aspect of the invention, the holder includes a handle, a first arm connected to the handle and configured to temporarily hold the medial tibial component, and a second arm connected to the handle and configured to temporarily hold the lateral tibial component.

In one version of this aspect of the invention, the first arm is pivotable with respect to the handle such that the medial tibial component can be adjusted in a transverse plane, and the second arm is pivotable with respect to the handle such that the lateral tibial component can be adjusted in the transverse plane.

In one version of this aspect of the invention, the first arm is pivotable with respect to the handle such that the medial tibial component can be adjusted in a sagittal plane, and the second arm is pivotable with respect to the handle such that the lateral tibial component can be adjusted in the sagittal plane.

These and other features, aspects, and advantages of the present invention will become better understood upon consideration of the following detailed description, drawings and appended claims.

### BRIEF DESCRIPTION OF THE DRAWINGS

FIG. 1A is a perspective view of a unicompartmental implant.

FIG. 1B is a coronal view of a bi-unicompartmental implant.

FIG. 1C is a coronal view of a bi-cruciate retaining implant.

FIG. 2A is a perspective view of a prior art tibial insert, along with sagittal and coronal cross-sections.

FIG. 2B is a perspective view of a tibial insert according to one embodiment of the invention, along with three sagittal cross-sections.

FIG. 2C is a perspective view of a tibial insert according to another embodiment of the invention, along with two sagittal cross-sections.

FIG. 2D is a perspective view of a tibial insert according to another embodiment of the invention, along with three coronal cross-sections.

FIG. 3 is a coronal view of a prior art tibial cutting block.

FIG. 4A is a coronal view of cutting guides of a tibial cutting block according to one embodiment of the invention.

FIG. 4B is a sagittal view of the cutting guides of FIG. 4A.

FIG. 4C is a sagittal view of the cutting guides of FIG. 4A with a saw blade and native tibia T.

FIG. 4D is a sagittal view of the cutting guides of FIG. 4A with lateral and medial slopes in the resected tibia T.

FIG. 5 is a coronal view of cutting guides of a tibial cutting block according to another embodiment of the invention.

FIG. 6A is a perspective view of cutting guides of a tibial cutting block according to another embodiment of the invention.

FIG. 6B is a transverse view of the cutting guides of FIG. 6A adjacent a tibia T.

FIG. 7A is a perspective view (bottom) of cutting guides of a tibial cutting block according to another embodiment of the invention, along with an axial view (top) of the cutting guides adjacent a tibia.

FIG. 7B is another perspective view (bottom) and another axial view (top) of the cutting guides of FIG. 7A.

FIG. 8 is a coronal view of cutting guides of a tibial cutting block according to another embodiment of the invention

FIG. 9A is a coronal view of cutting guides of a tibial cutting block according to another embodiment of the invention.

FIG. 9B is a coronal view of cutting guides of a tibial cutting block according to another embodiment of the invention.

FIG. 9C is a coronal view of cutting guides of a tibial cutting block according to another embodiment of the invention.

FIG. 9D is a cross-sectional view taken along line 9D-9D of FIG. 9A.

FIG. 9E is a cross-sectional view taken along line 9E-9E of FIG. 9B.

FIG. 9F is a cross-sectional view taken along line 9F-9F of FIG. 9C.

FIG. 10 is a coronal view of cutting guides of a tibial cutting block according to another embodiment of the invention adjacent a tibia T.

FIG. 11A is a transverse view of an instrument according to one embodiment of the invention for holding medial and lateral tibial components in a pre-determined position relative to each other.

FIG. 11B is a transverse view of the instrument of FIG. 11A holding medial and lateral tibial components in another pre-determined position relative to each other.

FIG. 12A is a transverse view of an instrument according to another embodiment of the invention for holding medial and lateral tibial components in a pre-determined position relative to each other.

FIG. 12B is a sagittal view of the instrument of FIG. 12A.

FIG. 13A is a transverse view of an instrument according to another embodiment of the invention for holding medial and lateral tibial components in a pre-determined position relative to each other.

FIG. 13B is a transverse view of the instrument of FIG. 13A holding medial and lateral tibial components in another pre-determined position relative to each other.

FIG. 14 is a transverse view of an instrument according to one embodiment of the invention for creating holes/slots in medial and lateral tibial compartments.

FIG. 15A is a coronal view of a tibia resected with a rounded edge of the invention compared to a tibia resected with a rectangular edge.

FIG. 15B is a coronal view of a tibia resected with a chamfered edge of the invention compared to a tibia resected with a rectangular edge.

FIG. 16 is a transverse view of a prior art tibial component.

FIG. 16A is a sagittal view of the prior art tibial component of FIG. 16 along line 16A-16A of FIG. 16.

FIG. 16B is a sagittal view of a tibial component according to one embodiment of the invention.

FIG. 16C is a sagittal view of a tibial component according to another embodiment of the invention.

FIG. 17A is a transverse view of prior art tibial components on a tibia.

FIG. 17B is a transverse view of tibial components according to one embodiment of the invention.

FIG. 17C is a transverse view of tibial component with an anterior bridge according to one embodiment of the invention.

FIG. 17D is a transverse view of tibial components according to another embodiment of the invention.

FIG. 17E is a transverse view of tibial components according to another embodiment of the invention.

FIG. 17F is a transverse view of tibial components according to another embodiment of the invention.

FIG. 18A shows anterior perspective, posterior perspective, and axial views of a prior art resected tibia.

FIG. 18B shows anterior perspective, posterior perspective, and axial views of a resected tibia according to one embodiment of the invention.

FIG. 19A is a sagittal view of a resected tibia having prior art anterior tibia bone removal, anterior tibia bone removal according to one embodiment of the invention, and anterior tibia bone removal according to another embodiment of the invention.

FIG. 19B is a perspective view of a tibial component according to one embodiment of the invention.

FIG. 19C is a perspective view of a tibial component according to another embodiment of the invention.

FIG. 19D is a perspective view of a tibial component according to another embodiment of the invention.

FIG. 19E is a cross-sectional view of the tibial component of FIG. 19B taken along lines 19E-19E of FIG. 19B.

FIG. 19F is a cross-sectional view of the tibial component of FIG. 19C taken along lines 19F-19F of FIG. 19C.

FIG. 20 shows sagittal and transverse views of a tibial component, along with sagittal (A-A) and coronal (B-B) cross-sections of a prior art tibial component (center) and a tibial component according to one embodiment of the invention (right).

FIG. 21A is a sagittal view of a femoral component.

FIG. 21B is a cross-sectional view of a femoral component, taken along line A-A of FIG. 21A.

FIG. 22A is a cross-sectional view of a femoral component with a modified mesial edge of the lateral condyle according to one embodiment of the invention.

FIG. 22B is a cross-sectional view of a femoral component with a modified mesial edge of the lateral condyle according to another embodiment of the invention.

FIG. 22C is a cross-sectional view of a femoral component with a modified mesial edge of the lateral condyle according to another embodiment of the invention.

FIG. 22D is a cross-sectional view of a femoral component with a modified mesial edge of the lateral condyle according to another embodiment of the invention.

FIG. 23A is a sagittal view of a femoral component with a modified condyle thickness according to one embodiment of the invention.

FIG. 23B is a sagittal view of a femoral component with a modified condyle thickness according to another embodiment of the invention.

FIG. 23C is a sagittal view of a femoral component with a modified condyle thickness according to another embodiment of the invention.

FIG. 23D is a sagittal view of a femoral component with a modified condyle thickness according to another embodiment of the invention.

FIG. 23E is a sagittal view of a femoral component with a modified condyle thickness according to another embodiment of the invention.

FIG. 23F is a sagittal view of a femoral component with a modified condyle thickness according to another embodiment of the invention.

FIG. 24 is an anterior perspective view of a femoral component according to one embodiment of the invention.

FIG. 24A is a transverse view of the femoral component of FIG. 24, taken along plane A-A of FIG. 24.

FIG. 24B is a transverse view of a femoral component according to another embodiment of the invention, taken along plane A-A of FIG. 24.

FIG. 24C is a transverse view of a femoral component according to another embodiment of the invention, taken along plane A-A of FIG. 24.

FIG. 25A is a sagittal view of a femoral component.

FIG. 25B is a transverse view of a femoral component, along line A-A of FIG. 25A.

FIG. 26A is a transverse view of a femoral component with a modified mesial edge of the medial condyle according to one embodiment of the invention.

FIG. 26B is a transverse view of a femoral component with a modified mesial edge of the medial condyle according to another embodiment of the invention.

FIG. 26C is a transverse view of a femoral component with a modified mesial edge of the medial condyle according to another embodiment of the invention.

FIG. 26D is a transverse view of a femoral component with a modified mesial edge of the medial condyle according to another embodiment of the invention.

FIG. 27A is a sagittal view of a femoral component.

FIG. 27B is a sagittal view of a femoral component with a modified condyle thickness according to an embodiment of the invention.

FIG. 27C is a sagittal view of a femoral component with a modified condyle thickness according to another embodiment of the invention.

FIG. 27D is a sagittal view of a femoral component with a modified condyle thickness according to another embodiment of the invention.

FIG. 27E is a sagittal view of a femoral component with a modified condyle thickness according to another embodiment of the invention.

FIG. 27F is a sagittal view of a femoral component with a modified condyle thickness according to another embodiment of the invention.

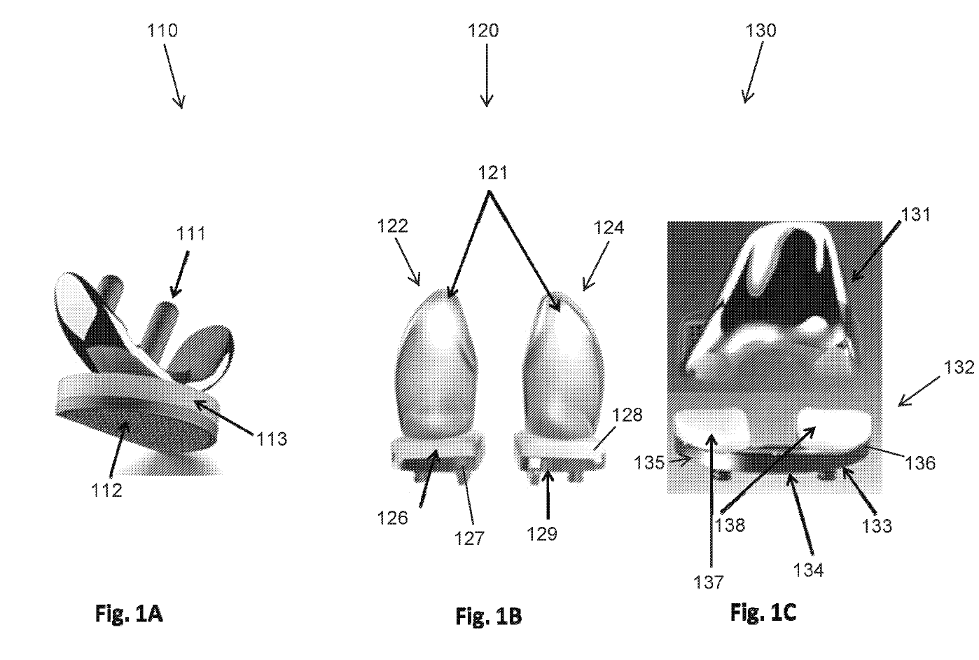

Like reference numerals will be used to refer to like or similar parts from Figure to Figure in the following description.

### DETAILED DESCRIPTION OF INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, while the invention is described in terms of knee implant and knee instrument designs that retain or permit the retention of the anterior cruciate ligament, these designs may also be used for knee implants and instruments that do not retain or do not permit the retention of the anterior cruciate ligament. The definitions of various terms used to describe the present invention are provided below.

### Definitions

The term “native” is used herein to imply natural or naturally occurring in the body. Examples of native structures include musculoskeletal structures such as the tibia bone (or tibia), femoral bone (or femur), tendon, muscle, ligament, etc.

The term “implant” is used herein to refer to a prosthetic component designed to augment or replace one or more native structures of the body. For example, a knee implant refers to a prosthetic component designed to augment or replace one or more native structures of the knee.

The term “slope” or “posterior slope” is used herein to refer to an angle relative to a tibial long axis measured in a sagittal plane. For example, “slope” or “posterior slope” of a tibial component is the angle between a surface, such as superior or inferior surface, and a tibial mechanical axis projected onto a sagittal plane.

The term “long axis” used herein in relation to the tibia bone or a femoral bone and refers to an axis parallel to the length of the bone such as an “anatomical” or “mechanical” axis. The “anatomical” axis refers to a line drawn along the length of the intramedullary canal of the bone. The “mechanical” axis of the femur bone refers to a line joining the center of the femoral head to a point where the “anatomic” axis meets intercondylar notch. The “mechanical” axis of the tibia refers to a line joining the medial tibial spine to the center of the ankle.

The term “resection depth” or “depth” refers to distance between native surface of a bone, such as tibia, and a cut surface of the bone generally measured in a superior-inferior direction.

The term “tibial eminence” or “tibial spine” is used herein to refer to the native structure of the proximal tibia between the medial and lateral articular surfaces which includes a central prominence and the attachment sites of the anterior cruciate ligament, posterior cruciate ligament, and the menisci.

The term “mesial” when used in reference to an implant refers to a portion of an implant situated near or directed towards the median or middle plane of the native bone when the implant is mounted on the native bone.

The terms “coronal”/“frontal”, “sagittal” and “transverse”/“axial” planes as used herein refer to anatomical “coronal”/“frontal”, “sagittal” and “transverse”/“axial” planes of the body or the native anatomical structure such as the tibia or femur. The descriptions of form, function or position of an implant or instrument with reference to such planes are intended to represent the form, function or position of the implant or instrument when it is placed/positioned against the anatomical structure in a generally intended manner.

### Tibial Articular Surface Geometry

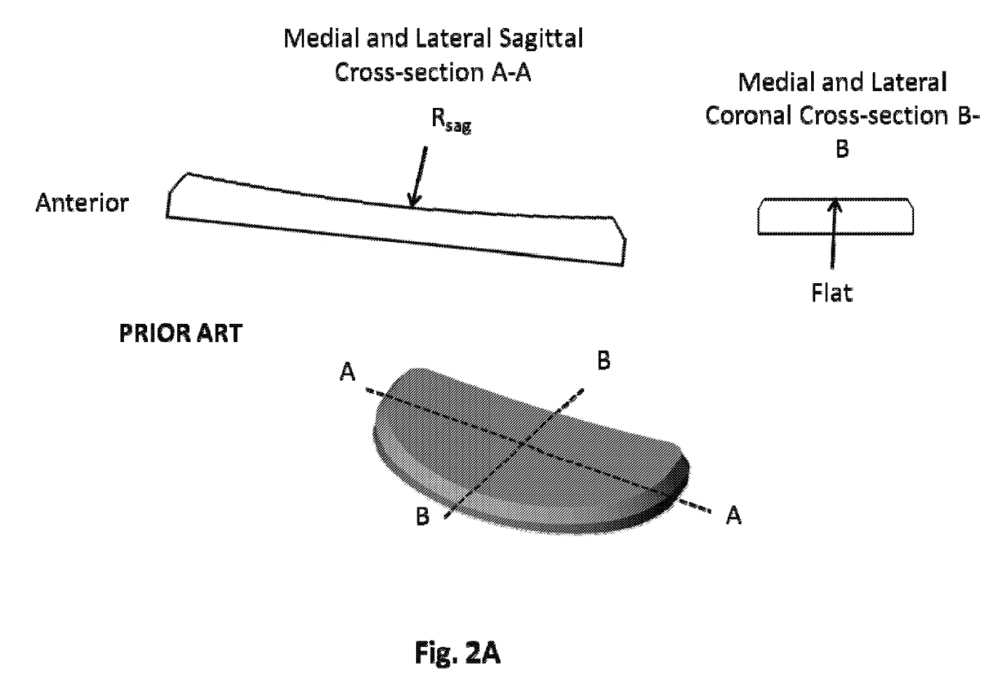

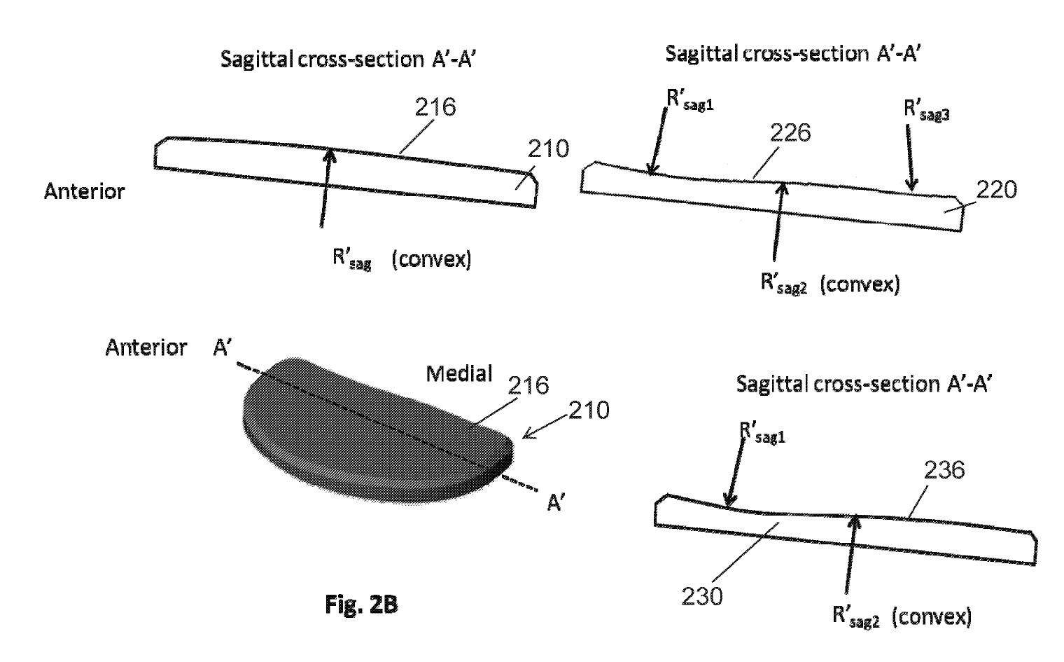

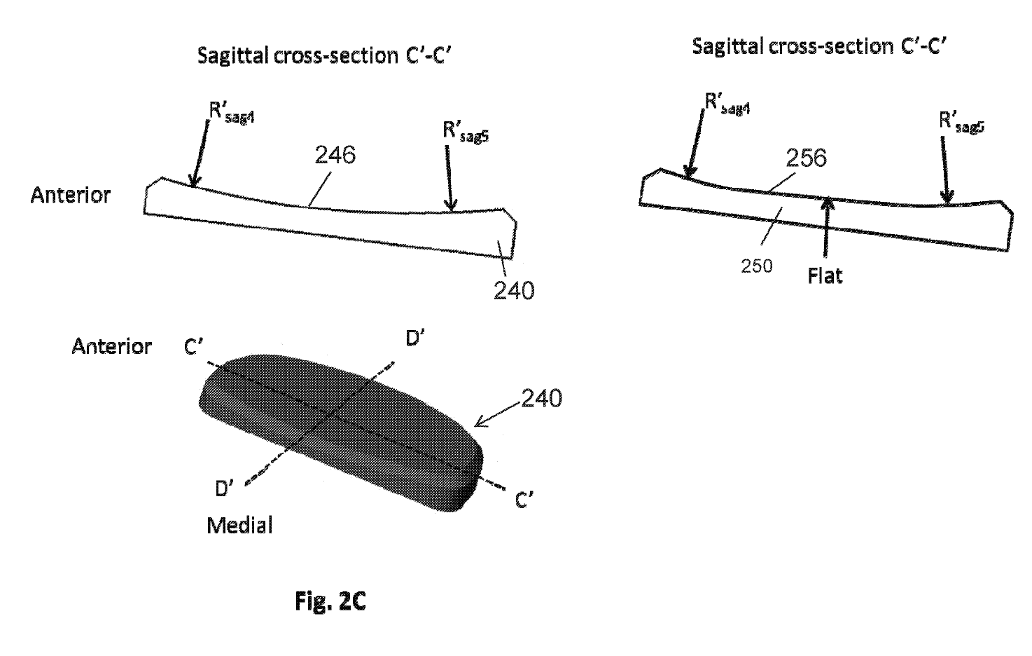

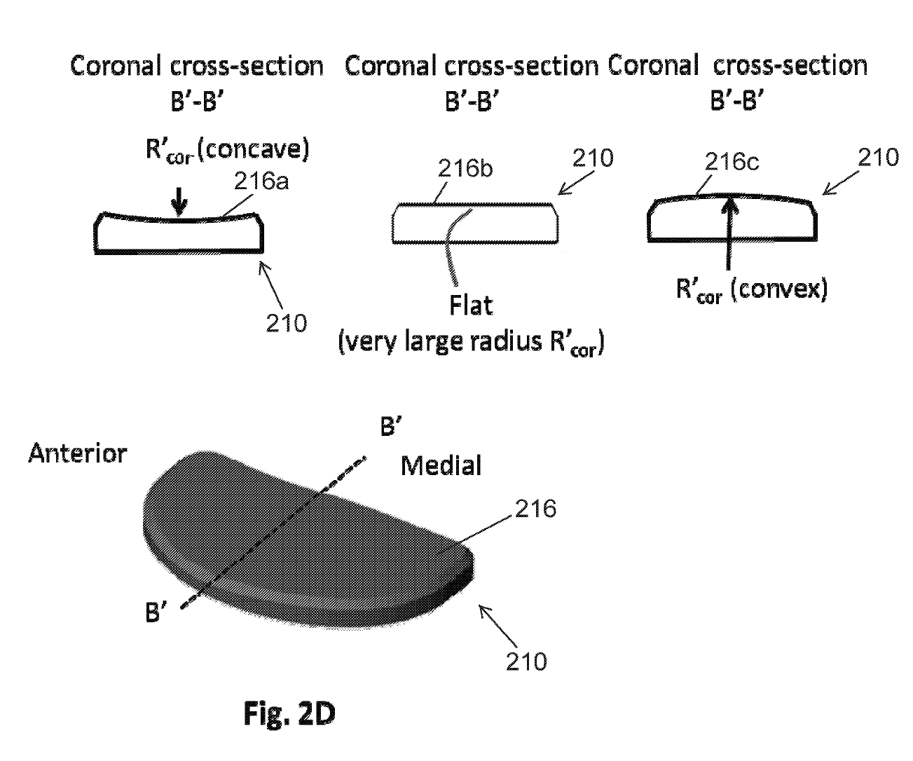

In contemporary Uni, Bi-Uni, and BCR implants, a sagittal plane cross-section of both the medial and lateral tibial articular surfaces of the tibial insert has a concave geometry composed of a single radius R_{sag} of about 200 mm, and a coronal plane cross-section has a concave or flat geometry (see FIG. 2A). In one embodiment of the invention, the medial or lateral tibial articular surface 216 of the tibial insert 210 has a convex sagittal geometry (i.e., sagittal plane cross-section geometry) composed of a convex radius (R′_{sag}, FIG. 2B). In another embodiment of the invention, the medial or lateral tibial articular surface 226 of the tibial insert 220 has a sagittal geometry composed of an anterior concave radius (R′_{sag1}), a central convex radius (R′_{sag2}) and a posterior concave radius (R′_{sag3}). In another embodiment of the invention, the medial or lateral tibial articular surface 236 of the tibial insert 230 has a sagittal geometry composed of an anterior concave radius (R′_{sag1}), and a central-posterior convex radius (R′_{sag2}). In preferred embodiments, R′_{sag1} has a value of about 60 mm, R′_{sag} and R′_{sag2} have a value of about 100 mm, and R′_{sag3} has a value of about 60 mm. However, these radii (R′_{sag}, R′_{sag1}, R′_{sag2}, R′_{sag3}) can range from 10 mm to 300 mm, 30 mm to 200 mm, 50 mm to 100 mm, etc.

In another embodiment of the invention, the medial or lateral tibial articular surface 246 of the tibial insert 240 has a concave sagittal geometry (i.e. sagittal plane cross-section geometry) composed of an anterior concave radius (R′_{sag4}) and a different posterior concave radius (R′_{sag5}, FIG. 2C). In preferred embodiments, R′_{sag4} has a value of about 100 mm, and R′_{sag5} has a value of about 130 mm. However, these radii (R′_{sag4}, R′_{sag5},) can range from 10 mm to 300 mm, 30 mm to 200 mm, 50 mm to 100 mm, etc. In other embodiments, the medial or lateral tibial articular surface 256 of the tibial insert 250 can have a generally concave sagittal geometry composed of one of more concave radii and/or flat sections/straight line (see FIG. 2C).

Further, the coronal geometry (i.e., coronal plane cross-section geometry) of the medial/lateral articular surface 216a, 216b. 216c of the tibial insert 210 may be concave (216a), flat (216b), or convex (216c) composed of one or more radii and/or flat sections (FIG. 2D). The radii (e.g. R′_{cor}) can range from 10 mm to 300 mm, 30 mm to 200 mm, 50 mm to 100 mm, etc.

### Instrumentation for Accurate Preparation of Tibial Bone

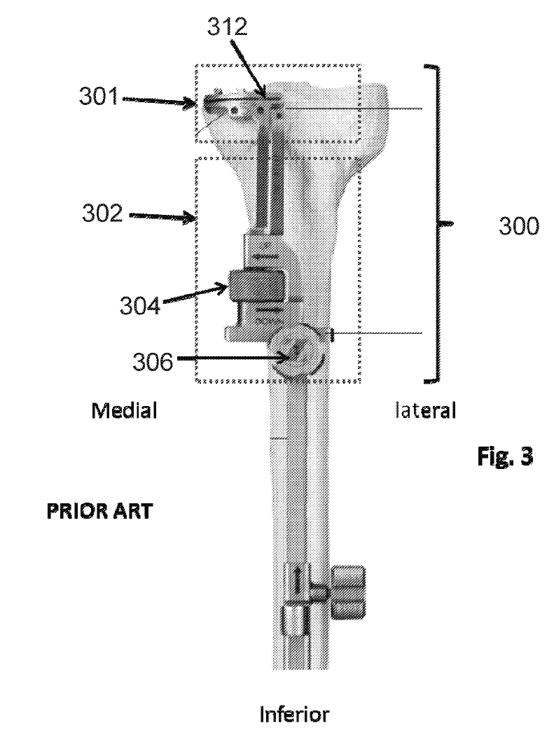

During preparation of the tibial bone for a Uni implant, a unicompartmental tibial cutting block 300 is used to resect the bone at the desired resection depth and posterior slope (see FIG. 3). In the prior art tibial cutting block shown in FIG. 3, the tibial cutting guide 301 can be adjusted in mediolateral (ML) and superoinferior (SI) direction relative to the block base 302 via the ML adjustment knob 304 and SI adjustment knob 306. The tibial cutting guide 301 includes a transverse cutting slot 312. With such a prior art instrumentation, in a Bi-Uni surgical procedure, the unicompartmental tibial cutting block 300 is independently positioned for the medial and lateral compartments to cut the bone in each compartment at the desired resection depth and posterior slope. However, due to manufacturing tolerances of cutting instruments, and inaccuracies in their placement, the desired position of the medial and lateral tibial components relative to each other may not be achieved. For example, if it is desired to achieve 0° posterior slope for the medial component and 4° posterior slope for the lateral component, then with a ±2° variation in tibial slope due to inherent inaccuracies, the actual medial component posterior slope may range from −2° to 2°, and the actual lateral component posterior slope may range from 2° to 6°. Thus, the relative difference in medial and lateral tibial component posterior slopes may range from 0° to 8°, as opposed to desired value of 4°.

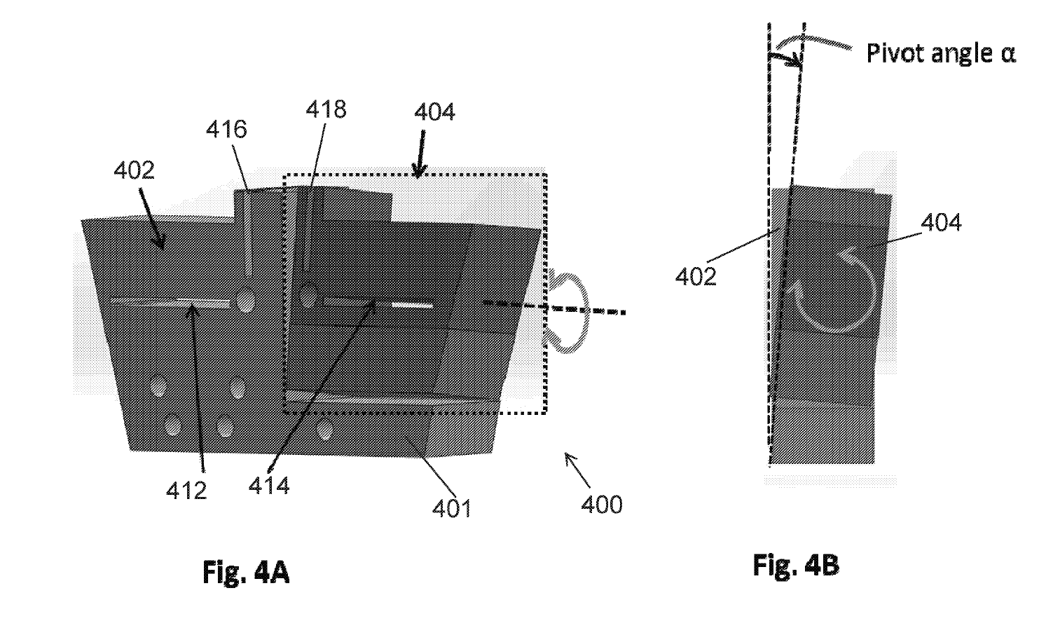

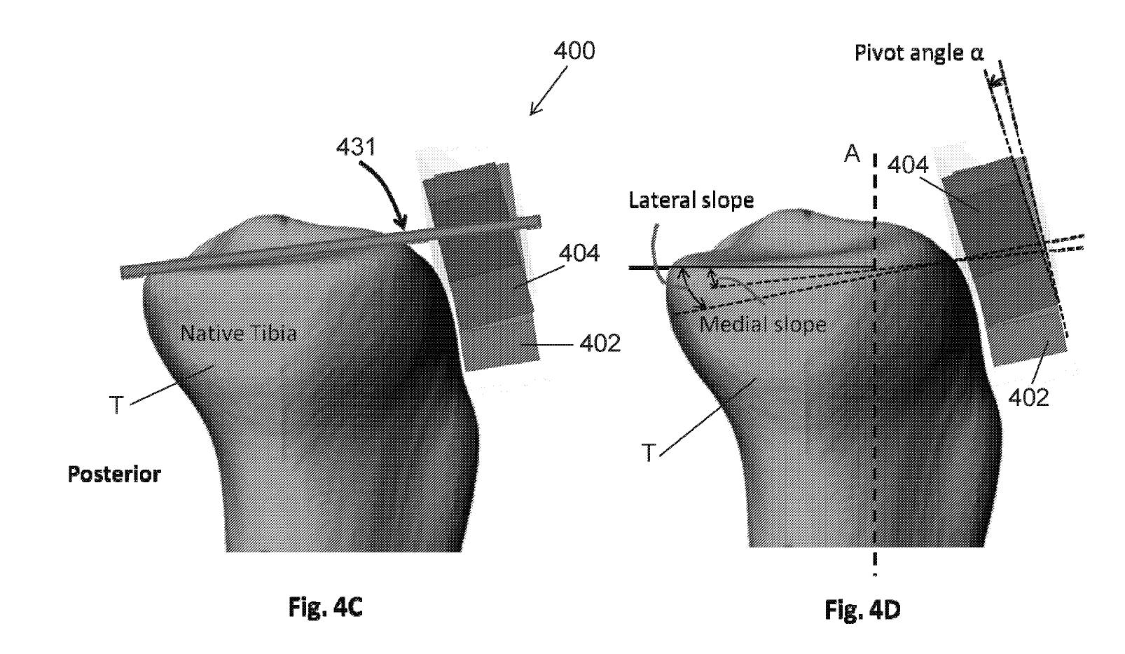

To address this, in one embodiment of the invention, the tibial cutting block 400 may comprise a medial cutting guide 402 and lateral cutting guide 404, wherein the medial cutting guide 402 or the lateral cutting guide 404 can pivot/rotate in a plane relative to the other side, such as in a sagittal plane when mounted on the tibia, to allow accurate control of medial/lateral slope relative to the fixed side (see FIGS. 4A and 4B). Pivoting can be achieved using a protrusion of the medial cutting guide 402 and/or the lateral cutting guide 404 that is rotatably mounted in a complementary hole in the base section 401 of the medial cutting guide 402. A saw blade or other such instrument passing through the transverse cutting slots 412, 414 and the tibial eminence cutting slots 416, 418 can then be used to cut the tibial bone. In the embodiment shown in FIGS. 4A and 4B, when the medial cutting guide 402 is fixed, the lateral cutting guide 404 can be pivoted as shown to obtain desired posterior slope relative to the medial side. During surgery, a saw blade 431 or other instrument slid through a slot on the fixed side, and resting on the anterior and posterior margins of the tibia, may be used to position the cutting block at the appropriate slope for the fixed side (medial side in FIG. 4C). The medial/lateral cutting guide may then be pivoted in a sagittal plane to obtain the desired posterior slope relative to the fixed side. This pivot angle α of the medial/lateral cutting guide relative to the fixed side may range from −10° to 10°, −5° to 5°, −3° to 3° etc. (see FIG. 4D where the tibial mechanical axis is labeled A).

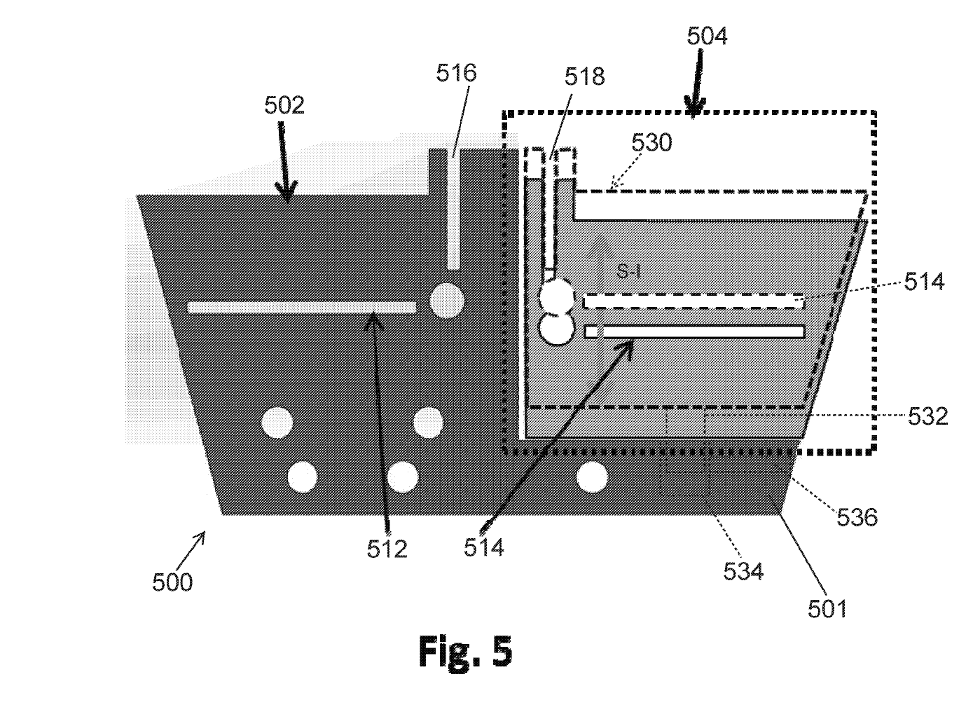

In another embodiment, the tibial cutting block 500 may comprise a medial cutting guide 502 and a lateral cutting guide 504, wherein the medial or lateral cutting guide can slide in a plane relative to the fixed side, such as along a superior-inferior direction S-I in a sagittal or coronal plane when mounted on the tibia, to allow desired resection depth relative to the fixed side (see FIG. 5). For example, in the embodiment shown in FIG. 5, the medial cutting guide 502 is fixed, and lateral cutting guide 504 can be adjusted to an adjusted position 530 to obtain desired resection depth on the lateral side relative to the medial side. Adjustment can be achieved using a protrusion 532 of the lateral cutting guide 504 that is slidably mounted in a complementary hole 534 in the base 501. A set screw 536 can secure the lateral cutting guide 504 in the adjusted position 530. In another embodiment, the lateral cutting guide 504 is fixed and the medial cutting guide 502 can be adjusted to obtain desired resection depth on the lateral side relative to the medial side. A saw blade or other such instrument passing through the transverse cutting slots 512, 514 and the tibial eminence cutting slots 516, 518 can be used to cut the tibial bone.

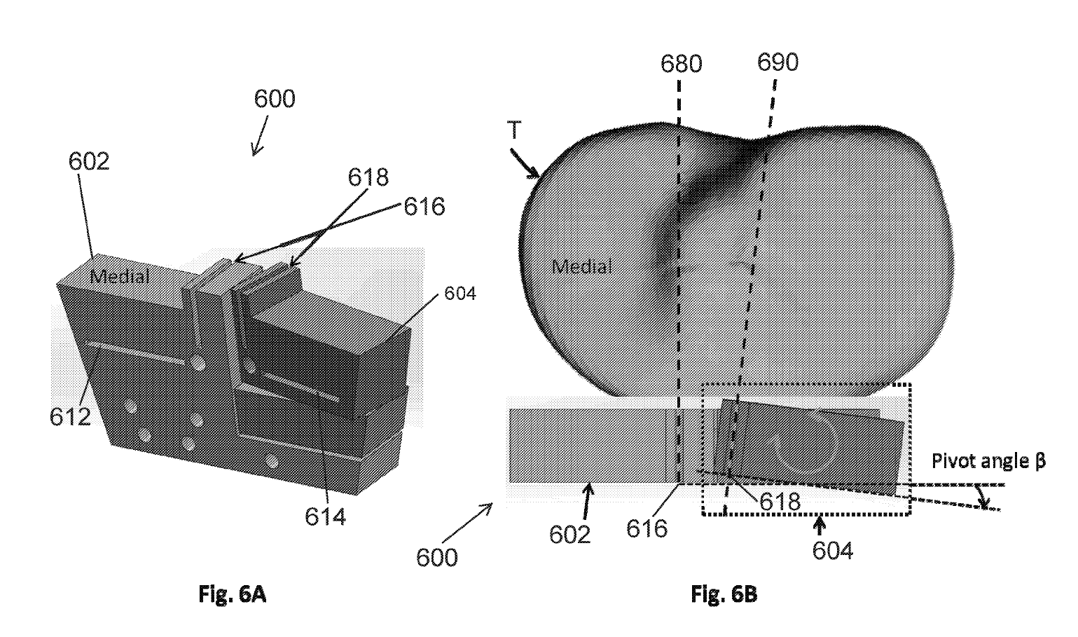

In another embodiment, the tibial cutting block 600 may comprise a medial cutting guide 602 and a lateral cutting guide 604, wherein the medial or lateral cutting guide can pivot in a plane relative to the fixed side, such as a transverse plane when mounted on the native tibia T, to allow resection of the tibial eminence along a desired direction relative to the fixed side (see FIGS. 6A and 6B). In the embodiment shown in FIGS. 6A and 6B, the medial cutting guide 602 is fixed, and lateral cutting guide 604 can pivot in a transverse plane. In another embodiment, the lateral cutting guide is fixed and the medial cutting guide can pivot in a transverse plane. Pivoting can be achieved using a protrusion of the cutting guide that is rotatably mounted in a complementary hole in the base as in the tibial cutting block 500. This pivot angle β of the medial/lateral cutting guide relative to the fixed side may range from −45° to 45°, −30° to 30°, −20° to 20°, −10° to 10° etc. A saw blade or other such instrument passing through the transverse cutting slots 612, 614 and the tibial eminence cutting slots 616, 618 can be used to cut the tibial bone. The tibial eminence cutting slots 616, 618 guide the creation of medial tibial eminence resection 680 and lateral tibial eminence resection 690.

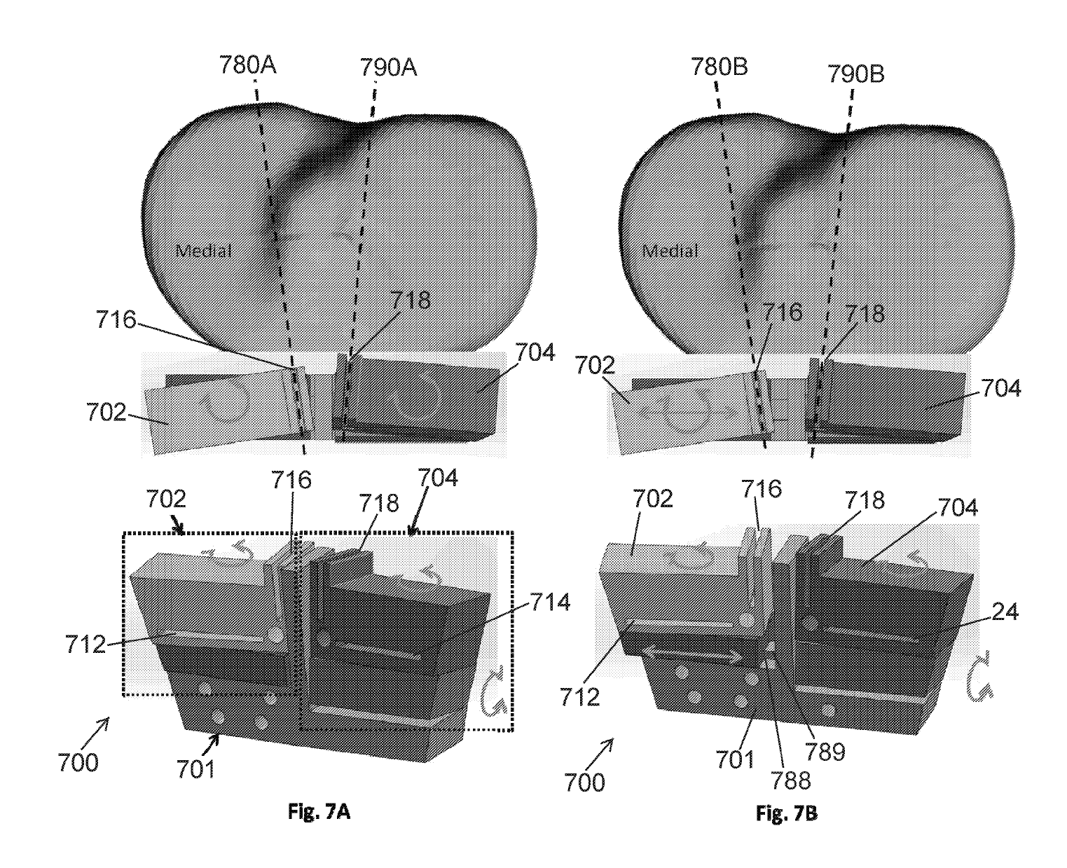

In another embodiment, the tibial cutting block 700 may comprise a medial cutting guide 702 and a lateral cutting guide 704, wherein the medial cutting guide 702 can pivot and slide in a transverse plane relative to a fixed block base 701, while the lateral cutting guide can pivot in a transverse and a sagittal plane relative to a fixed block base 701. See FIG. 7A and FIG. 7B. Pivoting can be achieved using a protrusion of the cutting guide that is rotatably mounted in a complementary hole in the base as in the tibial cutting block 500. Sliding can be achieved using a channel 788 of the cutting guide that is slidably mounted on a complementary rib 789 in the base 701. In another embodiment, the tibial cutting block may comprise a medial and a lateral cutting guide, wherein the lateral cutting guide can pivot and slide in a transverse plane relative to a fixed block base, while the medial cutting guide can pivot in a transverse and sagittal plane relative to a fixed block base. This would allow desired relative posterior slope between medial and lateral sides, and relative positions of the medial and lateral tibial eminence wall resections (see FIG. 7A and FIG. 7B). A saw blade or other such instrument passing through the transverse cutting slots 712, 714 and the tibial eminence cutting slots 716, 718 can be used to cut the tibial bone. The tibial eminence cutting slots 716, 718 guide the creation of medial tibial eminence resection 780A and lateral tibial eminence resection 790A (FIG. 7A), or the creation of medial tibial eminence resection 780B and lateral tibial eminence resection 790B (FIG. 7B).

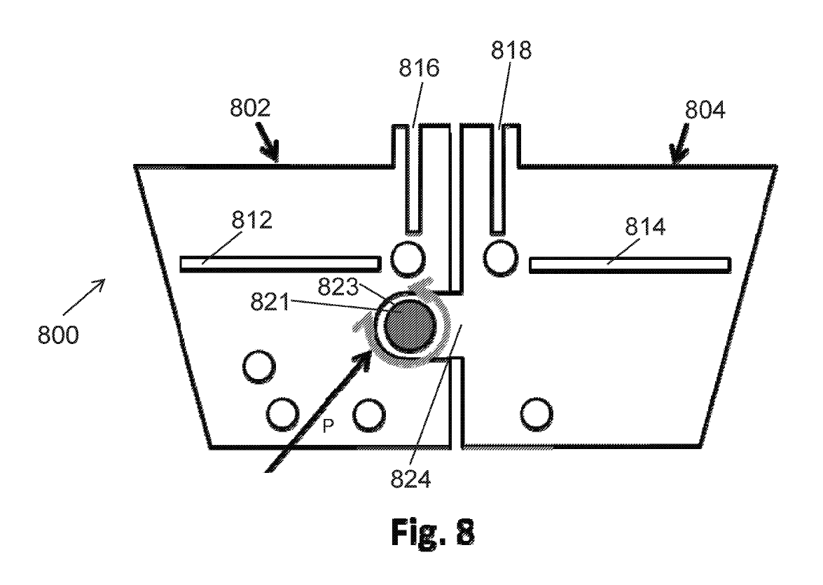

In another embodiment shown in FIG. 8, the tibial cutting block 800 may comprise a medial cutting guide 802 and a lateral cutting guide 804. A saw blade or other such instrument passing through the transverse cutting slots 812, 814 and the tibial eminence cutting slots 816, 818 can be used to cut the tibial bone. The medial cutting guide 802 can pivot as indicated at P in a coronal plane relative to the lateral cutting guide 804 by way of a pivot pin 821 rotatably positioned in a hole 823 of a tab 824 that extends from the lateral cutting guide 804. The pin 821 is connected to the medial cutting guide 802.

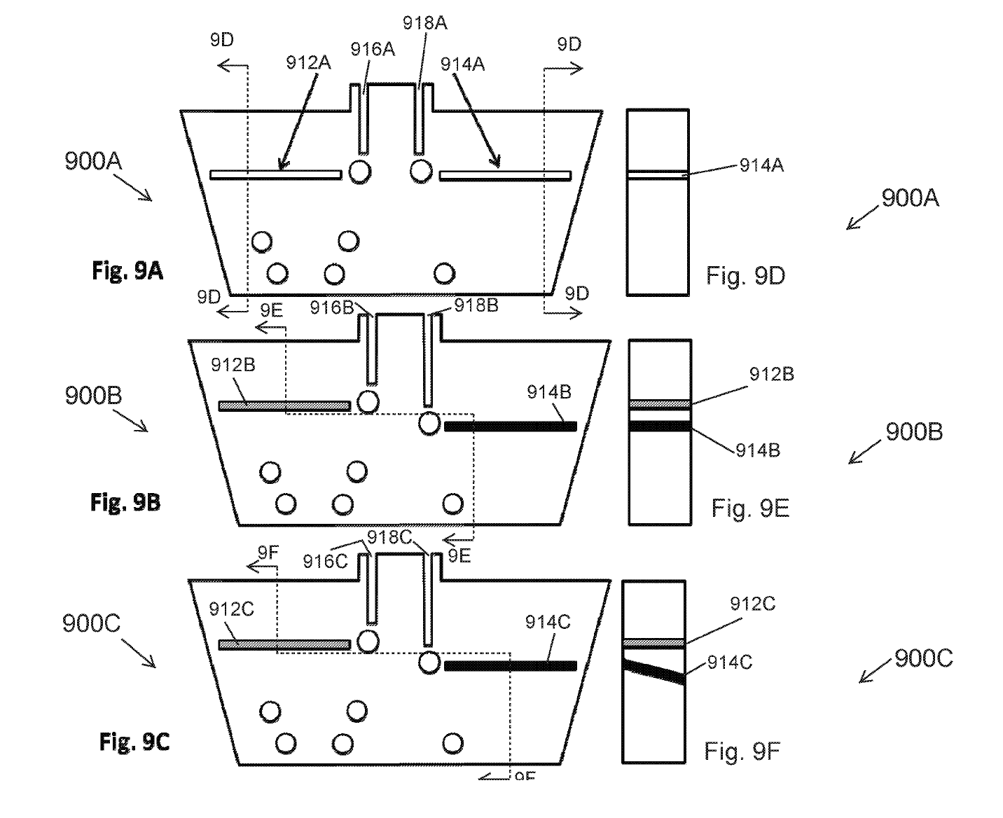

In another embodiment of FIGS. 9A-9F, a set including tibial cutting blocks 900A, 900B, 900C may be provided with medial and lateral transverse cutting slots having different relative resection depths and/or relative posterior slopes.

In FIGS. 9A and 9D, the tibial cutting block 900A comprises a medial transverse cutting slot 912A and a lateral transverse cutting slot 914A and tibial eminence cutting slots 916A, 918A. A saw blade or other such instrument passing through the transverse cutting slots 912A, 914A and the tibial eminence cutting slots 916A, 918A can be used to cut the tibial bone. The medial transverse cutting slot 912A and the lateral transverse cutting slot 914A provide equal resection depth and posterior slope.

In FIGS. 9B and 9E, the tibial cutting block 900B comprises a medial transverse cutting slot 912B and a lateral transverse cutting slot 914B and tibial eminence cutting slots 916B, 918B. A saw blade or other such instrument passing through the transverse cutting slots 912B, 914B and the tibial eminence cutting slots 916B, 918B can be used to cut the tibial bone. The medial transverse cutting slot 912B and the lateral transverse cutting slot 914B provide different resection depth and equal posterior slope.

In FIGS. 9C and 9F, the tibial cutting block 900C comprises a medial transverse cutting slot 912C and a lateral transverse cutting slot 914C and tibial eminence cutting slots 916C, 918C. A saw blade or other such instrument passing through the transverse cutting slots 912C, 914C and the tibial eminence cutting slots 916C, 918C can be used to cut the tibial bone. The medial transverse cutting slot 912C and the lateral transverse cutting slot 914C provide different resection depth and different posterior slope.

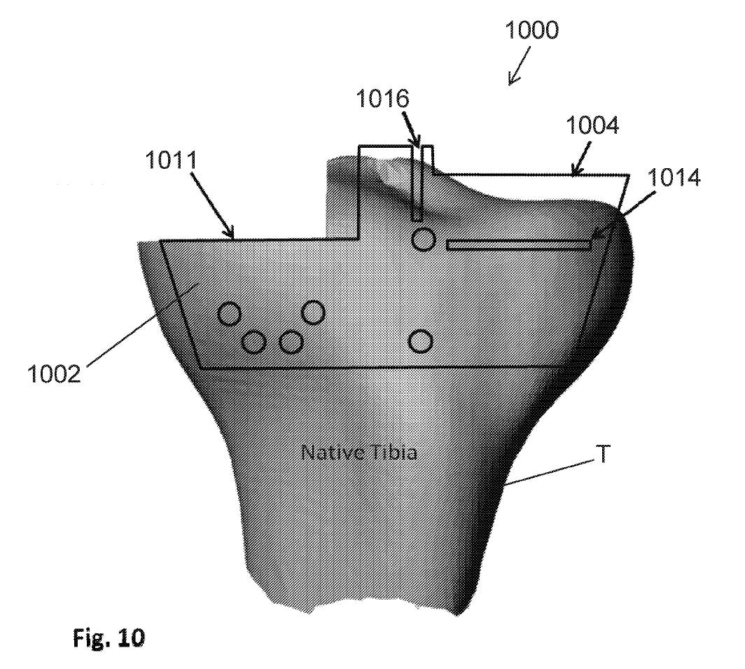

In some embodiments of the invention, the medial or lateral tibial bone can be cut first, and then a cutting guide or cutting block configured to use the cut tibial surface as reference may be used to guide the resection of the other side (medial or lateral). For example, in the embodiment shown in FIG. 10, the cutting block 1000 includes a lateral cutting guide 1004 having a lateral transverse cutting slot 1014 and a tibial eminence cutting slot 1016. The cutting block 1000 is configured to align a top edge 1011 of the medial cutting guide 1002 with the cut medial tibial surface as a reference to guide the resection of the lateral tibial compartment using a saw blade or other such instrument passing through the lateral transverse cutting slot 1014 and the tibial eminence cutting slot 1016.

In relation to the above inventions, it is understood that the location of the axis about which a cutting guide pivots/rotates or translates/shifts can differ from those shown in the specific non-limiting embodiments above.

### Instrumentation for Accurate Placement of Implants

In bi-unicompartmental surgery, it may be advantageous to maintain the desired relative positions of the medial and lateral tibial components during the surgical procedure. Therefore, in one embodiment of the invention, an instrument handle is provided to temporarily hold the medial and lateral tibial components in the desired relative positions, such as during cementing and/or seating of the components into the bone, to prevent or minimize relative shift in component positions from their desired or planned positions. The instrument handle can be detached from the medial and lateral tibial components prior to end of the surgical procedure. In another embodiment, an instrument handle is provided to temporarily hold the medial and lateral tibial trial components in the desired relative positions, such as for marking location of tibial implant fixation pegs or location of tibial eminence resections. This would aid in minimizing or preventing relative shift in final implant component positions from the desired or planned locations.

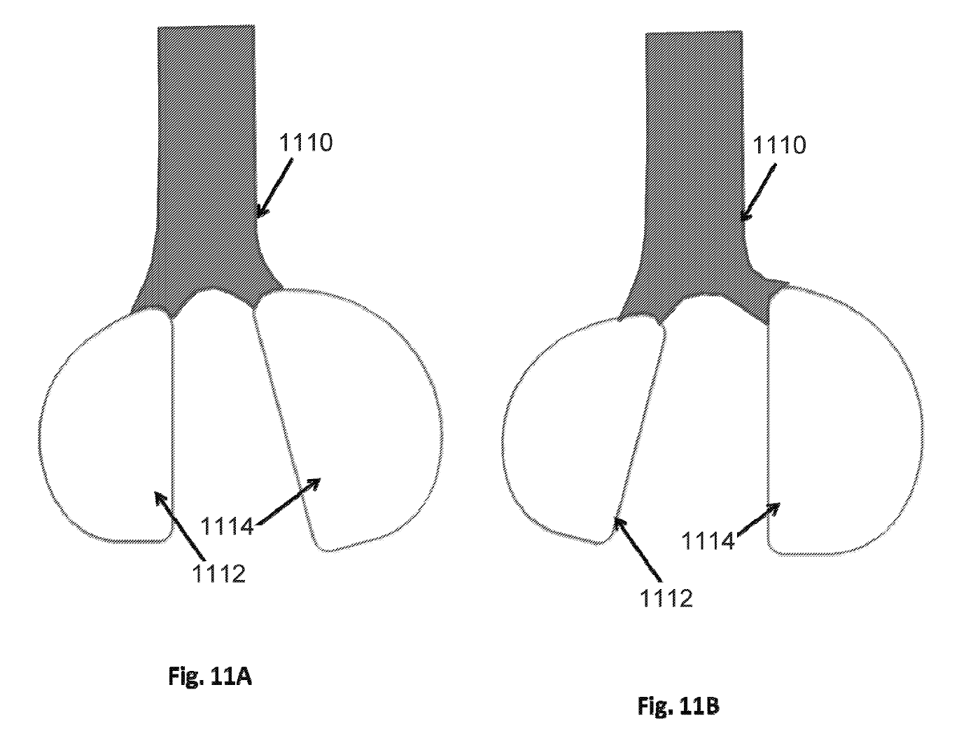

In one embodiment, the instrument handle 1110 is a single-piece component that is fabricated to hold the medial tibial component (implant or trial) 1114 and the lateral tibial component (implant or trial) 1112 (e.g., via clamping) in pre-determined positions relative to each other (see FIGS. 11A and 11B). This pre-determined position may also be unique for each patient and derived from pre-operative planning for that patient based on computed tomography, magnetic resonance, X-ray or other imaging techniques used to analyze the patient's native anatomy.

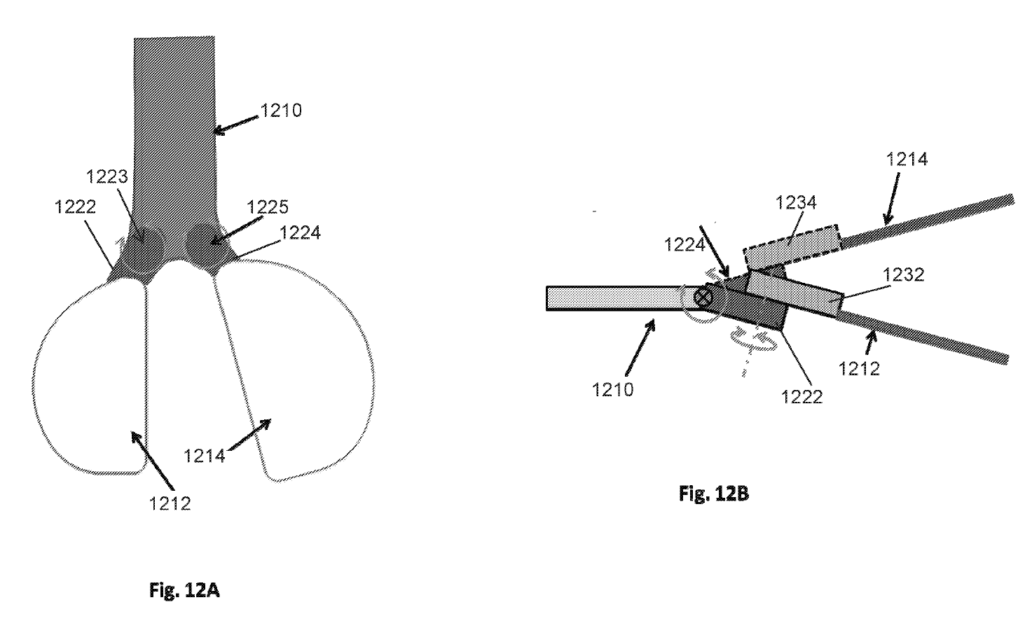

In other embodiments, an instrument handle 1210 can be configured during surgery, such as via aid of adjustable arms 1222,1224, to achieve desired orientation of the medial tibial component (implant or trial) 1214 and the lateral tibial component (implant or trial) 1212 in 3D space (see FIGS. 12A and 12B). Such rotational or translational adjustments within the instrument handle 1210 may be provided in one or more planes such as sagittal, transverse, or coronal planes. In the non-limiting example embodiment shown in FIG. 12A, the instrument handle 1210 allows relative orientation of the medial tibial component 1214 and the lateral tibial component 1212 to be adjusted in the transverse plane via pivot pins 1223,1225 of each of the adjustable arms 1222,1224. In the non-limiting example embodiment of FIG. 12B (lateral view), the instrument handle allows relative orientation of the medial and lateral tibial components to be adjusted in two perpendicular planes via a pivot pin of each of the adjustable arms 1222,1224 and a pivot pin of each of the adjustable arms 1232,1234.

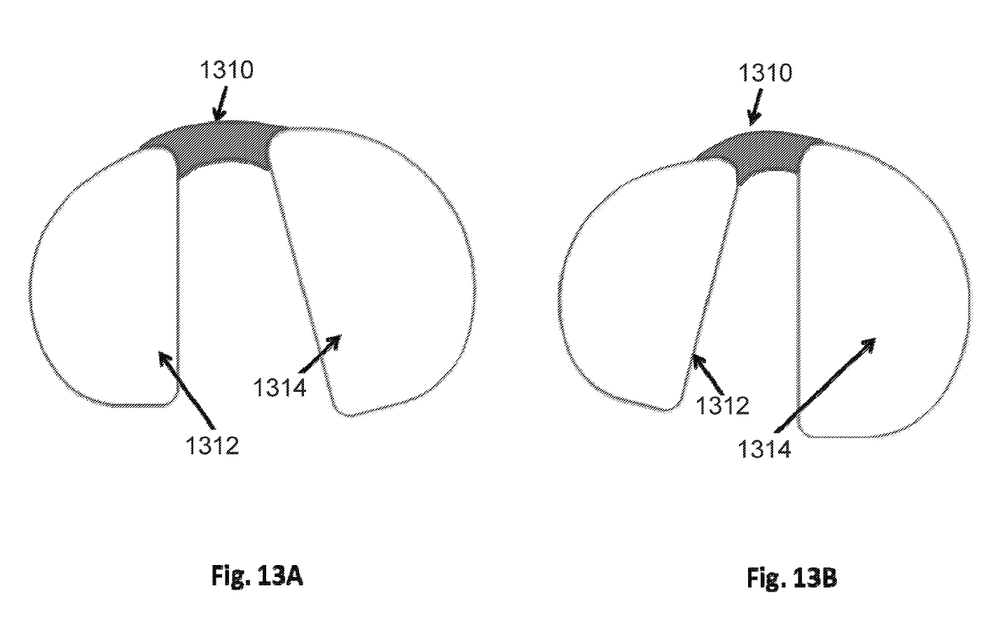

In another embodiment of the invention, a clip 1310 is provided to temporarily hold (e.g., via clamping) the medial tibial component (implant or trial) 1314 and the lateral tibial component (implant or trial) 1312 in pre-determined relative positions (see FIGS. 13A and 13B).

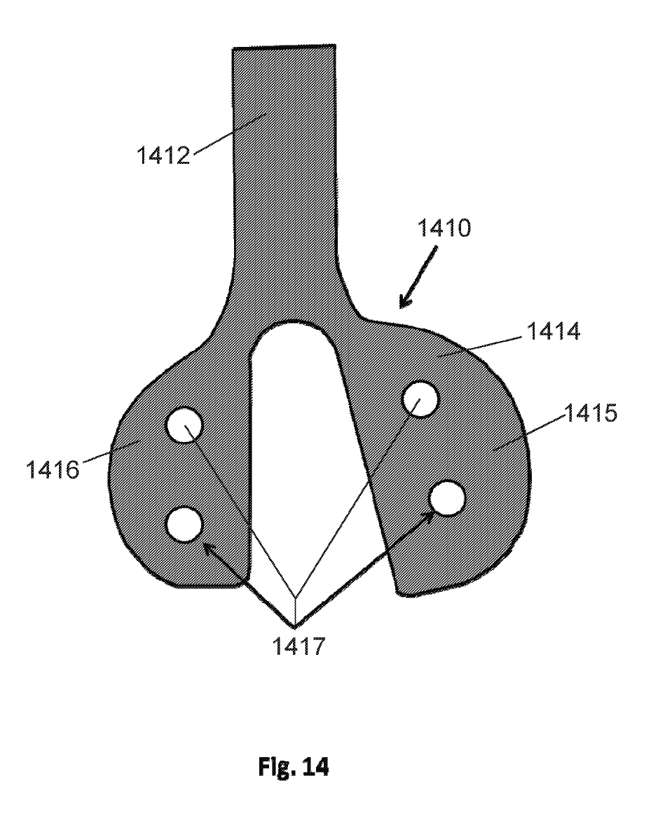

In another embodiment, a single-piece tibial trial component 1410 with a handle 1412 and trial tibial baseplate 1414 is provided, wherein the medial compartment 1415 and the lateral compartment 1416 are configured according to the desired location of the medial and lateral tibial implant components. The single-piece tibial trial may include features (tibial fixation peg guide holes 1417) to guide the creation of holes/slots in the tibial bone to receive fixation features such as fixation pegs or keels of the tibial implant component (see FIG. 14). The aforementioned single-piece instrument handles, clips, and tibial trial may be made specifically for individual patients according to desired relative orientation/position of the medial and lateral tibial components. The aforementioned single-piece instrument handles, clips, and tibial trial may attach to the tibial components at any location on the component, such as at an anterior location as shown in FIGS. 11-13, a central location, or a posterior location, etc.

### Tibial Component Design

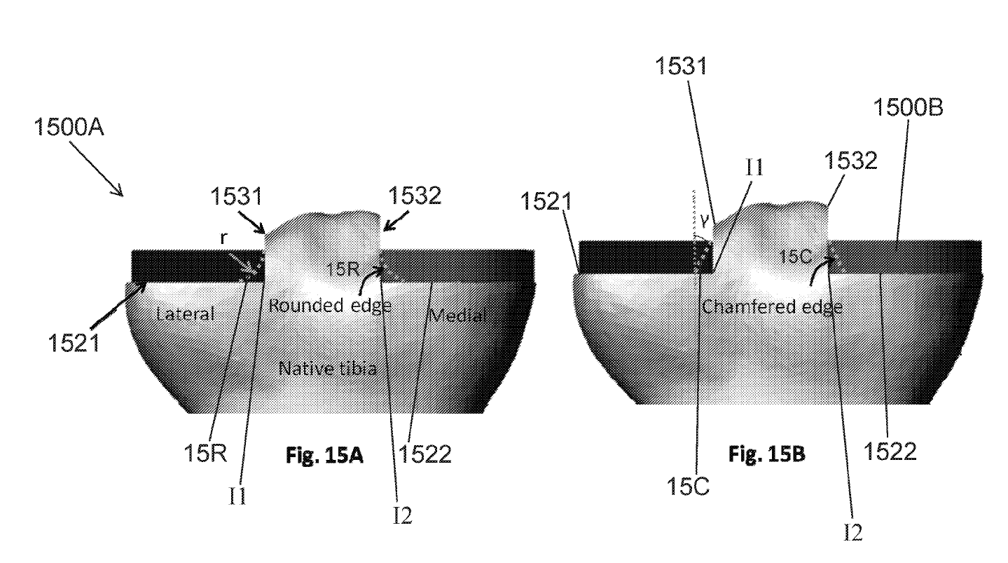

In conventional unicompartmental, bi-unicompartmental or bi-cruciate retaining surgical procedure, the medial/lateral surface of the native tibial eminence is resected perpendicular to the transverse tibial bone cuts to match the rectangular coronal geometry of conventional tibial components (see FIGS. 15A and 15B). Note the lateral transverse bone cut 1521 and medial transverse bone cut 1522 in FIG. 15A. The rectangular coronal geometry of conventional tibial components can cause an increase in stress within the tibial bone near the intersections I1 and I2 of the tibial eminence bone cuts 1531,1532 and the transverse tibial bone cuts 1521,1522 respectively, and potentially lead to fracture of the tibial bone. To address this, in one embodiment of the invention, a mesial edge (inner edge or edge closer to the tibial eminence) of the tibial component 1500A (tibial baseplate and/or insert) has a rounded coronal geometry (see 15R in FIG. 15A). The rounded edge 15R may have a radius r, ranging from 2 to 50 mm, 10 to 40 mm, 15 to 25 mm etc. In another embodiment, a mesial edge of the tibial component 1500B or tibial baseplate has a chamfered coronal geometry (see 15C in FIG. 15B). The chamfered edge 15C may have a chamfer angle γ, ranging from 2° to 75°, 15° to 50°, 25° to 40° etc. Corresponding to these embodiments, the tibial eminence bone may be prepared to have a rounded or chamfered coronal geometry at the intersection of the tibial eminence and transverse plane cuts. With these embodiments of the invention, a portion of compressive load acting on the tibial component 1500A or 1500B is transferred to bone at the intersections I1, I2 of the tibial eminence cut and transverse bone cut which may reduce risk of bone fracture.

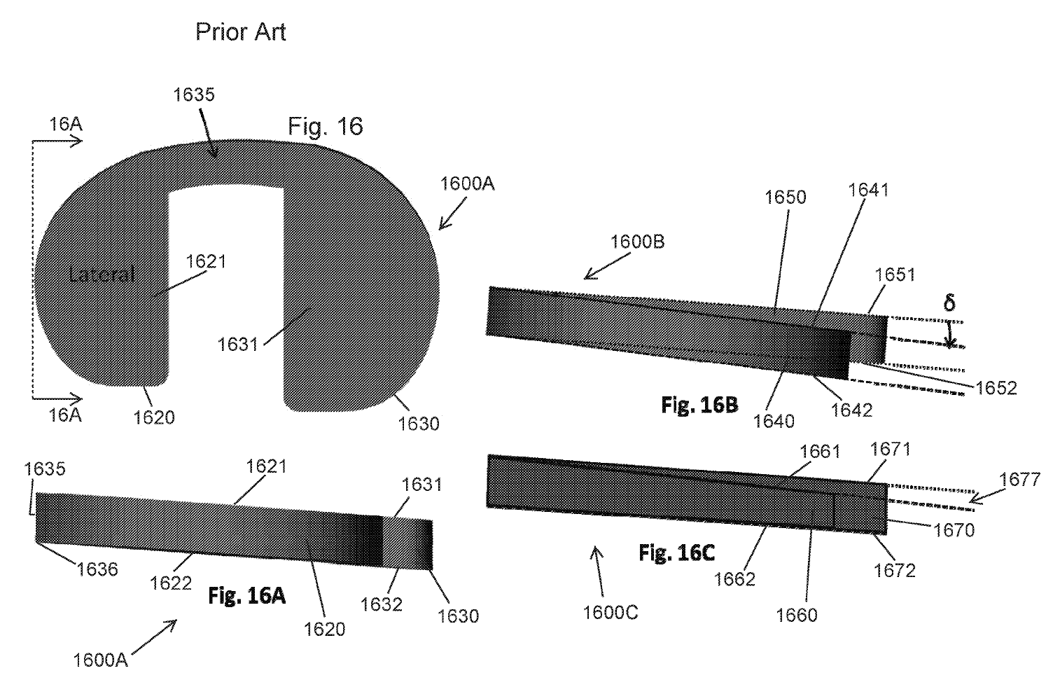

In a conventional BCR tibial component 1600A (tibial baseplate and/or tibial insert) shown in FIGS. 16 and 16A having an anterior bridge 1635, the proximal surface 1621 of the lateral side 1620 and the proximal surface 1631 of the medial side 1630 lie on the same plane, and the distal surfaces 1622, 1632 of the medial and lateral sides 1620,1630 lie on the same plane, i.e., the distal and proximal surfaces of the medial and lateral sides have the same posterior slope (see FIG. 16A). In contrast, in the native knee, the medial and lateral compartments have different slopes even in the same individual subject/patient. To accommodate this variation in the native anatomy, in one embodiment of the invention, the tibial component 1600B, shown in FIG. 16B, has an anterior bridge similar to 1635 of FIG. 16, and is designed such that the distal surface 1652 and the proximal surface 1651 of the medial side 1650 have a different posterior slope than the distal surface 1642 and proximal surface 1641 of the lateral side 1640. The difference in slopes (δ) can be about 5°, but can range from −15° to 15°, −10° to 10°, −5° to 5°, −2° to 2° etc. (see FIG. 16B). In another embodiment, the tibial component 1600C, shown in FIG. 16C, has an anterior bridge similar to 1635 of FIG. 16, and is designed such that the distal surface 1672 of the medial side 1670 and the distal surface 1662 of the lateral side 1660 have the same posterior slope, but the proximal surface 1671 of the medial side 1670 and the proximal surface 1661 and the lateral side 1660 have different posterior slopes (see FIG. 16C). The difference in slopes 1677 can be about 5°, but can range from −15° to 15°, −10° to 10°, −5° to 5°, −2° to 2° etc. Such implants may be manufactured from additive manufacturing processes such as laser sintering, 3D printing etc. These implants 1600B, 1600C may also be designed specifically for individual patients according to their native anatomies.

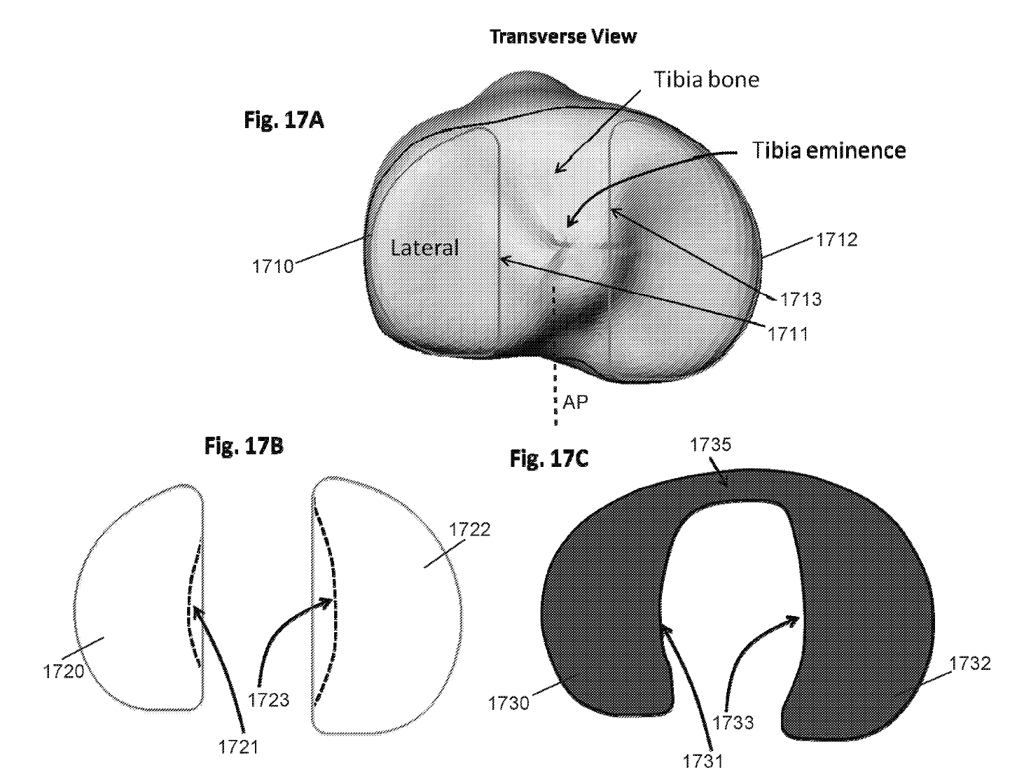

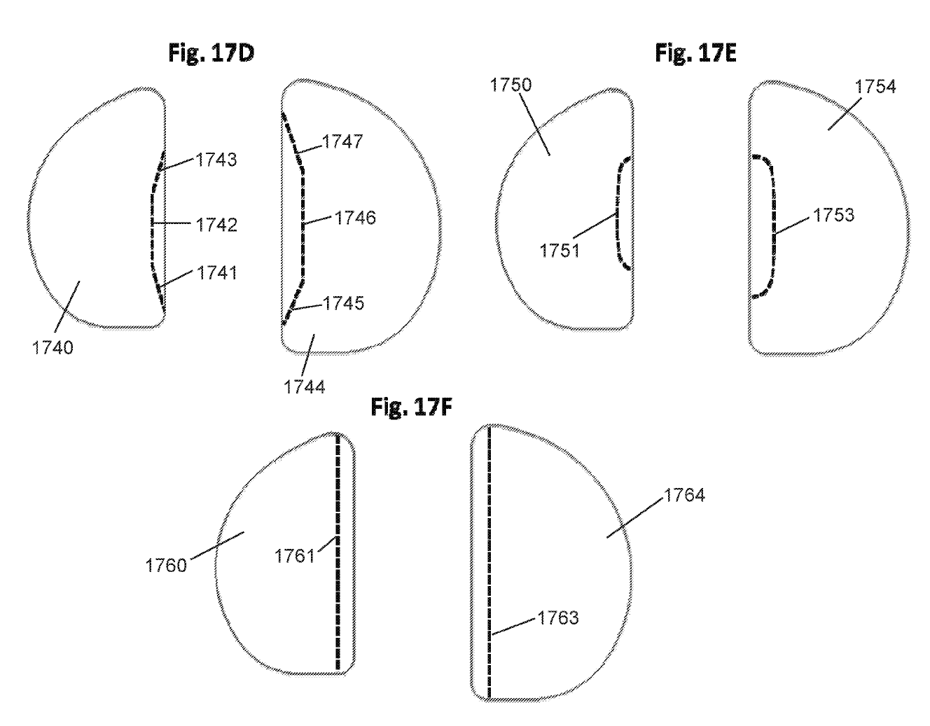

In a transverse plane, the conventional Uni, Bi-Uni and BCR lateral tibial component 1710 and medial tibial component 1712 have a straight mesial edges 1711, 1713 respectively (see FIG. 17A). Generally, when these tibial components 1710, 1712 are implanted in the knee, this straight mesial edges 1711,1713 of the tibial implant are parallel to the anteroposterior axis AP of the tibia. In contrast to this geometry of the conventional implants, the mesial boundary of the native tibial eminence in the transverse plane is not a straight line. Therefore, preparation of the tibial eminence to accommodate the straight edge of the conventional implant may result in excess removal of tibial eminence bone, which could increase the risk of tibial eminence bone fracture. To address this, in one embodiment of the invention, the mesial edge 1721 of the lateral tibial component 1720 and the mesial edge 1723 of the medial tibial component 1722 have an arcuate geometry in the transverse plane (see FIG. 17B). In another embodiment of the invention, the mesial edge 1731 of the lateral tibial component 1730 and the mesial edge 1733 of medial tibial component 1732 have an arcuate geometry in the transverse plane (see FIG. 17C). The lateral tibial component 1730 and the medial tibial component 1732 are attached via anterior bridge 1735. In mesial edges 1721, 1723, 1731, 1733, this arcuate geometry can extend over the entire mesial edge or a portion thereof.

In other embodiments, the arcuate geometry may be approximated with three straight lines 1741, 1742, 1743 for the lateral tibial component 1740 and three straight lines 1745, 1746, 1747 for the medial tibial component 1744 (see FIG. 17D).

In other embodiments, in a transverse plane, the mesial edge 1751 for the lateral tibial component 1750 and the mesial edge 1753 for the medial tibial component 1754 may include one or more convex/concave arcs, or multiple line segments (see FIG. 17E).

In other embodiments, the mesial edge 1761 for the lateral tibial component 1760 and the mesial edge 1763 for the medial tibial component 1764 may include a rectangular notch/cutout extending over a portion of the mesial edge (see FIG. 17F).

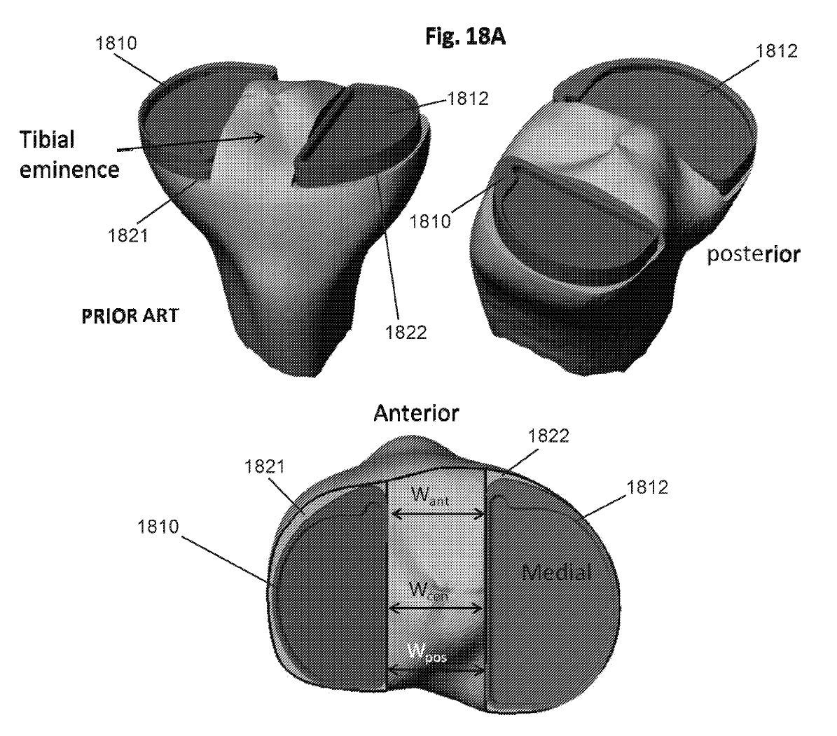

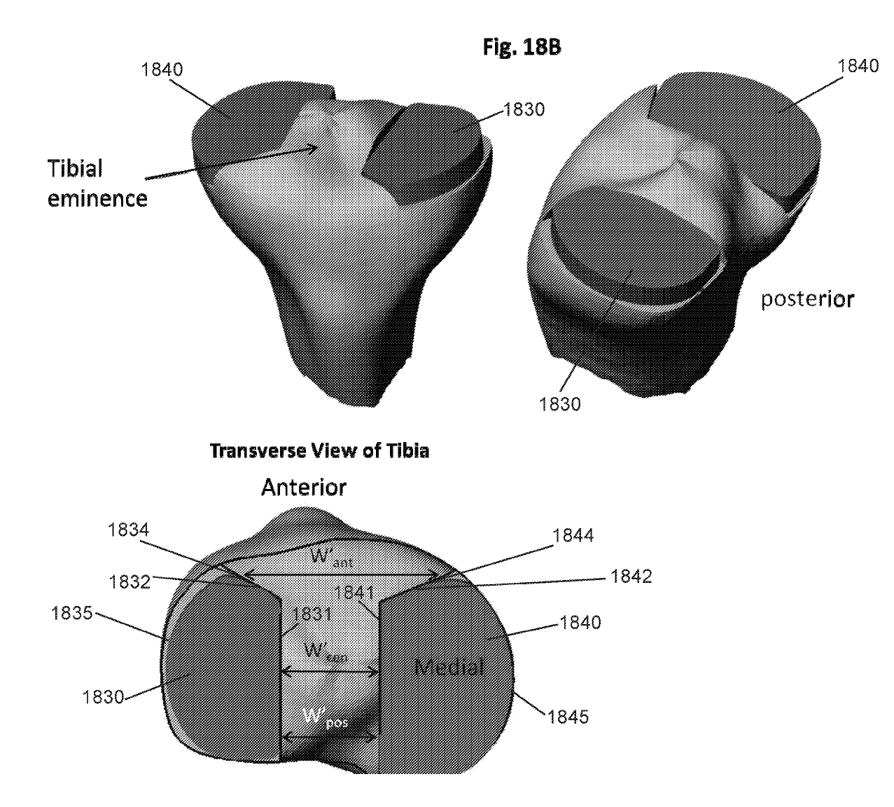

In conventional bi-unicompartmental knee surgery using a lateral tibial component 1810 and a medial tibial component 1812, the medial and/or lateral tibial bone is resected with a transverse cut going across/through the anteroposterior extent of the medial/lateral tibial compartment, and the tibial eminence is resected with a sagittal cut going across/through the anteroposterior extent of the tibial eminence (see FIG. 18A). This creates mediolateral widths of the tibial bone retained between the medial plateau 1822 and lateral plateau 1821 of wₐₙₜ, w_{cen} and wₚₒₛ (see the transverse view at the bottom of FIG. 18A). These bony resections may weaken the tibial eminence and pose the risk of fracture near the anterior margin of the eminence due to the load acting on the eminence from the anterior cruciate ligament. To address this, in one embodiment of the invention, the surgical preparation of the bone and implant design are configured to increase the amount of retained anterior tibial bone. This may be achieved, for example, by resecting the tibial eminence along multiple planes rather than a single plane and limiting the anteroposterior extent of the transverse medial and/or lateral tibial cut to preserve greater amount of the anterior tibial bone (see FIG. 18B). With this surgical technique and implant design, in the transverse plane, the mediolateral width of the tibial bone retained between the medial and lateral plateaus would be wider anteriorly than centrally or posteriorly (w′ₐₙₜ>w′_{cen} and/or w′ₐₙₜ>w′ₚₒₛ). In the embodiment of FIG. 18B, a lateral tibial component 1830 includes a mesial edge 1831 that joins a chamfered anterior edge 1832 that extends to an anterior point 1834 intermediate the mesial edge 1831 and the lateral edge 1835. A medial tibial component 1840 includes a mesial edge 1841 that joins a chamfered anterior edge 1842 that extends to an anterior point 1844 intermediate the mesial edge 1841 and the medial edge 1845.

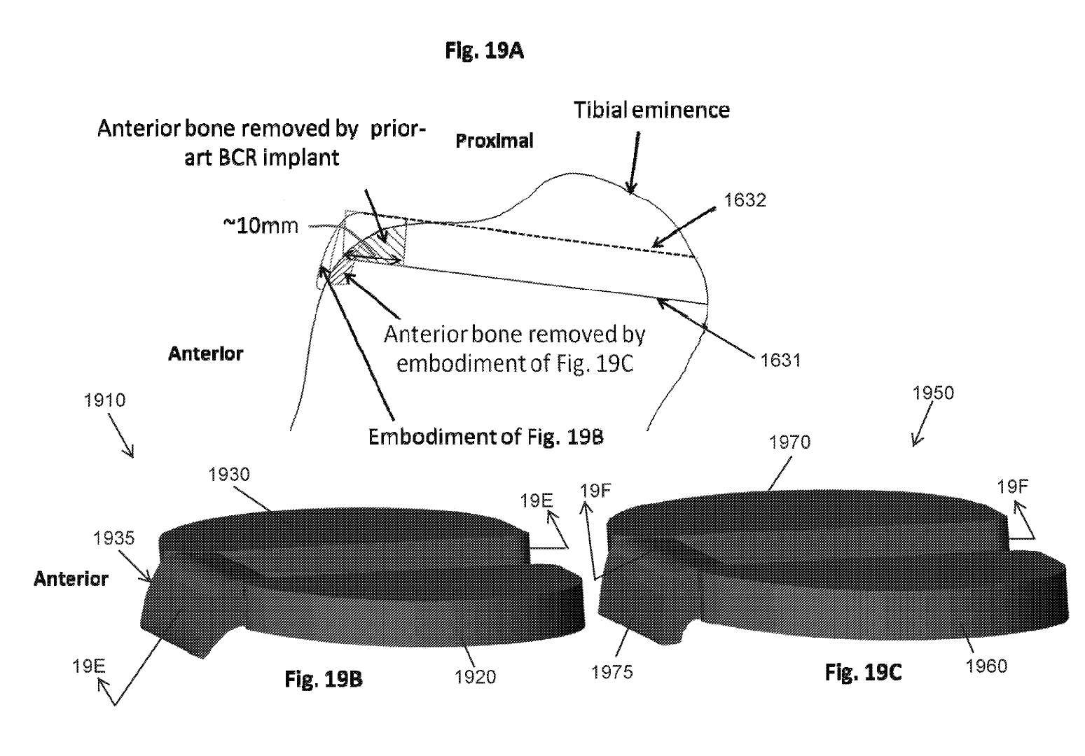

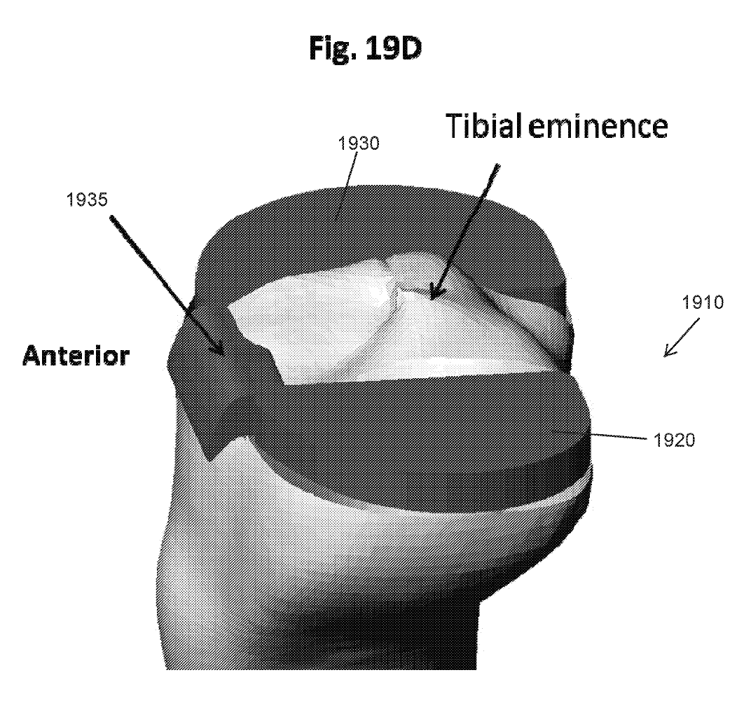

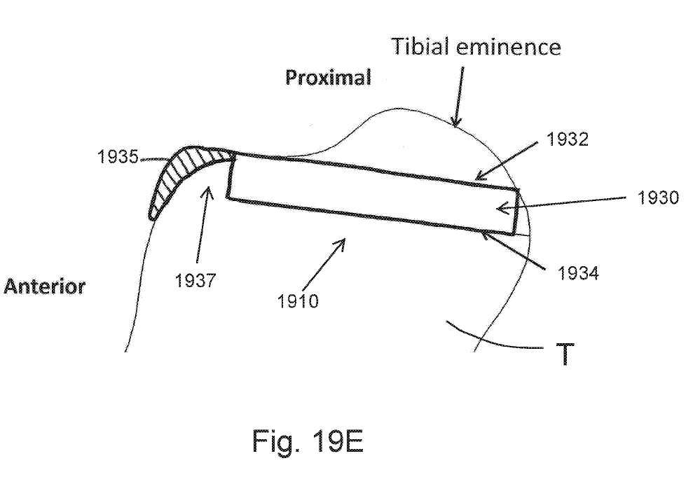

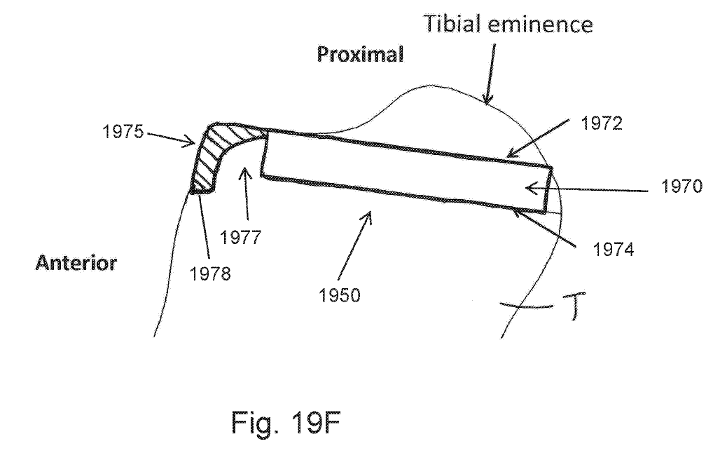

Looking at FIG. 16A, in a conventional BCR implant, the distal surface 1636 of the anterior-bridge 1635 is in the same plane as the distal surfaces 1622, 1632 of the medial and lateral side compartments 1620,1630. Consequently, tibial bone in the anterior portion of the tibial eminence has to be removed to accommodate the anterior-bridge of the tibial component. See FIG. 19A. The anteroposterior width of the tibial bone resection to accommodate the anterior-bridge is about 10 millimeters as shown in FIG. 19A. However, this removal of bone can reduce the strength of the eminence bone leading to risk of bone fracture. To address this, in one tibial component 1910 shown in FIGS. 19B, 19D, and 19E and another tibial component 1950 shown in FIGS. 19C and 19F, the anterior-bridge is configured such that a superior portion of the anterior-bridge drapes over the bone anterior to the tibial eminence, thereby creating a dome or cavity underneath the anterior bridge.

Referring to FIGS. 19B, 19D, and 19E, the tibial component 1910 includes a lateral tibial component 1930 and a medial tibial component 1920 connected by an anterior bridge 1935. The lateral tibial component 1930 has a proximal surface 1932 and a distal surface 1934. A superior portion of the anterior bridge 1935 drapes over the tibia bone anterior to the tibial eminence, thereby creating a cavity 1937 underneath the anterior bridge 1935.

Referring now to FIGS. 19C and 19F, the tibial component 1950 includes a lateral tibial component 1970 and a medial tibial component 1960 connected by an anterior bridge 1975. The lateral tibial component 1970 has a proximal surface 1972 and a distal surface 1974. A superior portion of the anterior bridge 1975 drapes over the tibia bone anterior to the tibial eminence, thereby creating a cavity 1977 underneath the anterior bridge 1975. Further, to achieve sufficient strength against failure of the anterior bridge 1975, a portion of the anterior bridge 1975 also extends below the level of the distal surface of the medial component 1960 and/or the lateral tibial component 1970. In the tibial component 1950, the distal portion 1978 of the anterior bridge 1975 is not in the same plane as the distal surfaces of the medial and lateral tibial components 1960,1970. Thus, in some embodiments of the present invention, the distal portion of the anterior bridge may not be in the same plane as the distal surfaces of the medial and lateral tibial components.

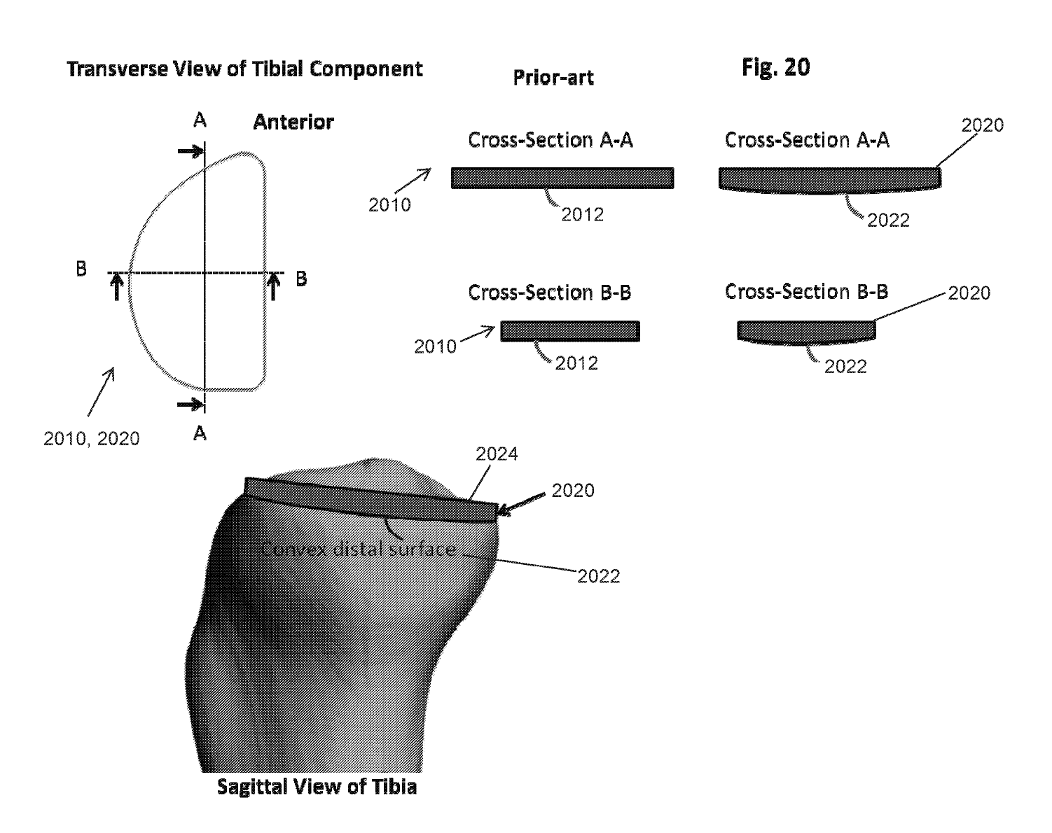

In conventional knee implants, the distal surface 2012 of the tibial component 2010 interfacing with the tibial bone has a flat/planar geometry, and the corresponding tibial bone cut is also planar (see FIG. 20). In one embodiment of the invention, a tibial component 2020 includes a distal surface 2022 (facing the tibial bone) having a convex geometry in the coronal plane A-A and the sagittal plane B-B (see FIG. 20). This convex geometry of the tibial component 2020 may interface with a concavity prepared in the tibial bone to better resist loosening caused by eccentric loading acting on the tibial component 2020. In another embodiment of the invention, the distal surface of the tibial component may have a concave geometry facing the tibial bone.

### Femoral Component Design:

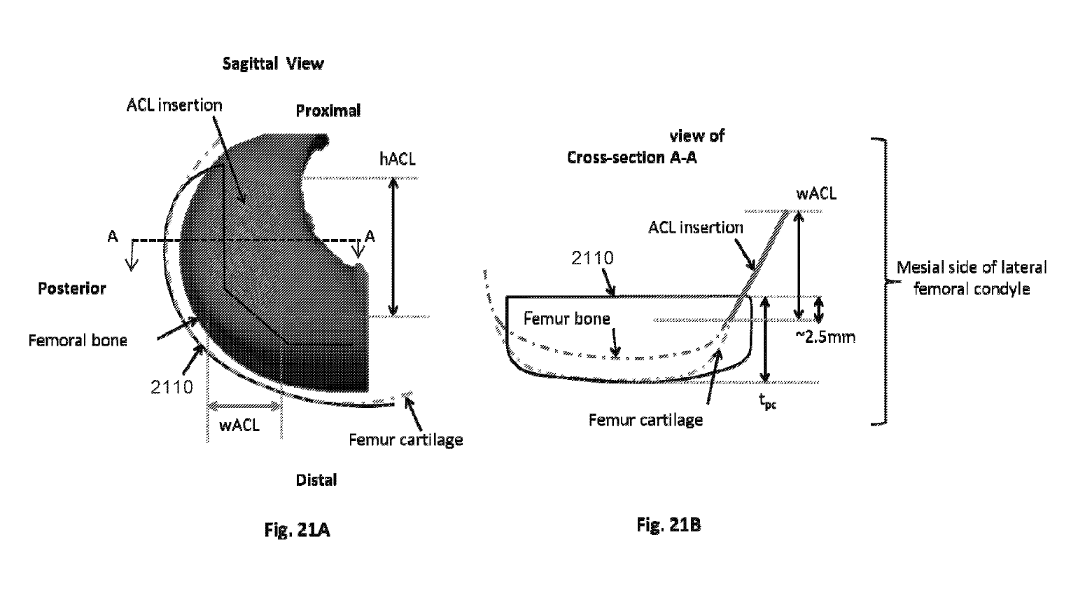

The insertion of the native ACL on the femoral side lies along the mesial side of the lateral condyle 2110 of the femoral component (see FIG. 21A). The ACL insertion in the average knee has a width wACL of about 11 mm, and a height hACL of about 20 mm. The posterior edge of the ACL insertion is approximately 6.5 mm from posterior femoral condyle cartilage surface, and approximately 14 mm from distal femoral condyle cartilage surface (see FIGS. 21A and 21B). In a conventional femoral component, the condyle thickness of the implant is greater than or equal to about 8 mm. Thus, removal of corresponding bone from the posterior condyle of the femur to accommodate the femoral implant can result in removal of significant proportion of the ACL insertions and/or ACL fibers at/near the insertion, thereby weakening the ligament.

To address this, in one set of embodiments, the geometry of the femoral component is modified to remove material along a mesial edge of the lateral condyle of the femoral component. In FIG. 22A, there is comparison of a conventional lateral condyle 2110 and a lateral condyle 2120 of the present invention in which a removal of material can be achieved through a gradual chamfer surface region 2122 at the mesial edge 2121. In FIG. 22B, there is comparison of a conventional lateral condyle 2110 and a lateral condyle 2130 of the present invention in which a removal of material can be achieved with a smooth convex arcuate profile surface region 2132 at the mesial edge 2131. In FIG. 22C, there is comparison of a conventional lateral condyle 2110 and a lateral condyle 2140 of the present invention in which a removal of material can be achieved a sharp concave curvilinear transition surface region 2142 in condyle thickness near the mesial edge 2141. In FIG. 22D, there is comparison of a conventional lateral condyle 2110 and a lateral condyle 2150 of the present invention in which a removal of material can be achieved a sharp concave transition surface region 2152 in condyle thickness near the mesial edge 2151. In preferred embodiments, the parameters wₐ and hₐ (shown in FIG. 22C) have a value of about 3 mm, but can range from 0.5 to 6 mm, 1 to 4 mm etc. The parameter wₐ can be measured perpendicularly from the mesial edge 2141 to a normal line N1 to a junction of the surface region and the inner surface 2143. The parameter hₐ can be measured perpendicularly from the inner surface 2143 to a normal line N2 to a junction of the surface region and the mesial edge, is in a range of range 0.5 to 7 millimeters.

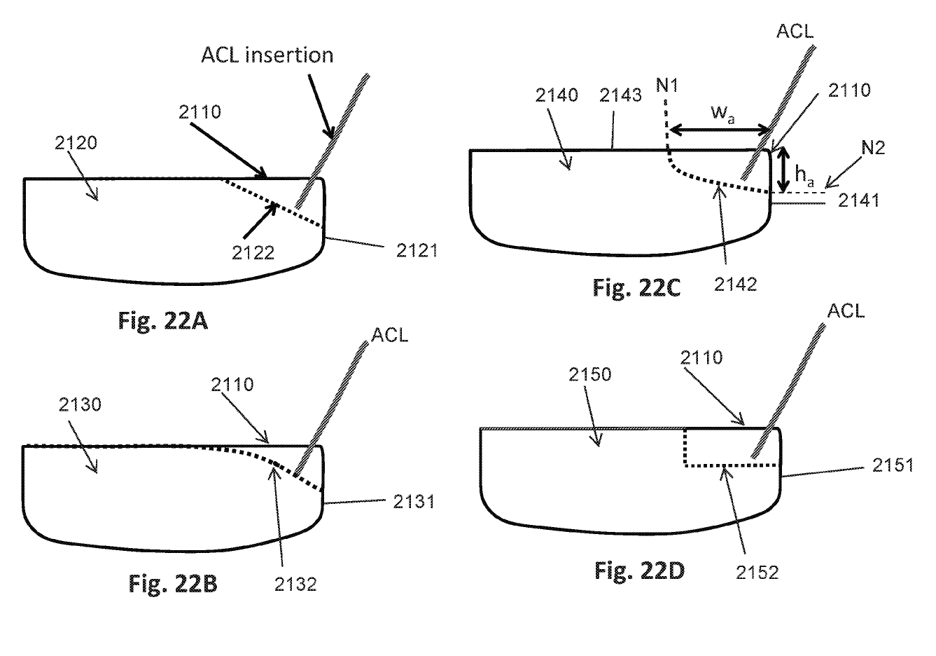

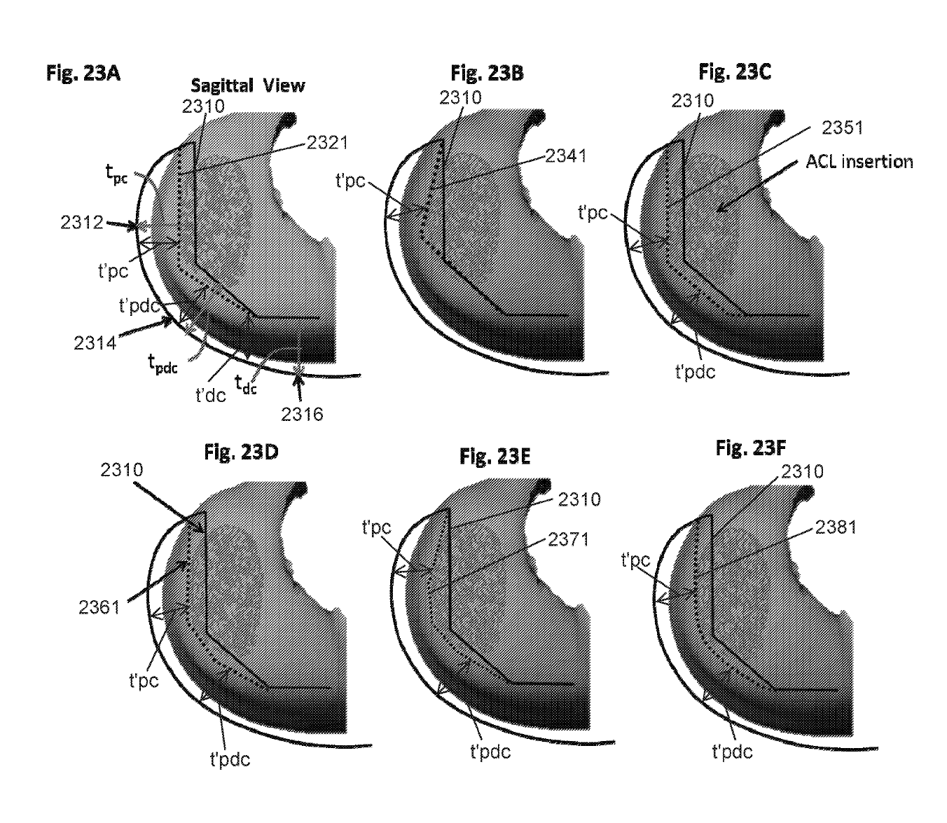

In a conventional femoral component, the posterior and distal condyle thickness (t_{pc} and t_{dc}) are generally equal and about 8 mm or greater (range 8 to 12 mm). In some embodiments of the invention, the thickness of the posterior femoral condyle may be reduced, resulting in modification of the sagittal plane geometry of the inner surface of the femoral implant interfacing with the femoral bone. In some embodiments (see FIGS. 23A-23F), the posterior (t′pc) and/or posterodistal condyle thickness (t′pdc) may be reduced to about 6.5 mm (range 1 mm to 7.5 mm), while the distal condyle thickness (t′dc) is greater and about 8 mm (range 8 mm to 15 mm). In other embodiments, the posterior (t′pc), posterodistal (t′pdc), and distal condyle thickness (t′dc) may be equal and about 6.5 mm (range 1 mm to 7.5 mm). In the above embodiments, the sagittal geometry of the inner surface of the posterior, posterodistal and distal condyle interfacing with the femoral bone may comprise one or more straight lines as in embodiments of FIGS. 23A-23E, or may be arcuate as in embodiment of FIG. 23F.

Specifically, in FIG. 23A, there is a comparison of a conventional lateral condyle 2310 (having posterior condyle 2312, posterodistal condyle 2314 and distal condyle 2316) and a lateral condyle of the present invention in which an inner surface 2321 (shown in dashed lines) creates reduced posterior condyle thickness (t′pc) and reduced posterodistal condyle thickness (t′pdc). In FIG. 23B, there is a comparison of a conventional lateral condyle 2310 and a lateral condyle of the present invention in which an inner surface 2341 (shown in dashed lines) creates reduced posterior condyle thickness (t′pc). In FIG. 23C, there is a comparison of a conventional lateral condyle 2310 and a lateral condyle of the present invention in which an inner surface 2351 (shown in dashed lines) creates reduced posterior condyle thickness (t′pc) and reduced posterodistal condyle thickness (t′pdc). In FIG. 23D, there is a comparison of a conventional lateral condyle 2310 and a lateral condyle of the present invention in which an inner surface 2361 (shown in dashed lines) creates reduced posterior condyle thickness (t′pc) and reduced posterodistal condyle thickness (t′pdc). In FIG. 23E, there is a comparison of a conventional lateral condyle 2310 and a lateral condyle of the present invention in which an inner surface 2371 (shown in dashed lines) creates reduced posterior condyle thickness (t′pc) and reduced posterodistal condyle thickness (t′pdc). In FIG. 23F, there is a comparison of a conventional lateral condyle 2310 and a lateral condyle of the present invention in which an inner surface 2381 (shown in dashed lines) creates reduced posterior condyle thickness (t′pc) and reduced posterodistal condyle thickness (t′pdc).

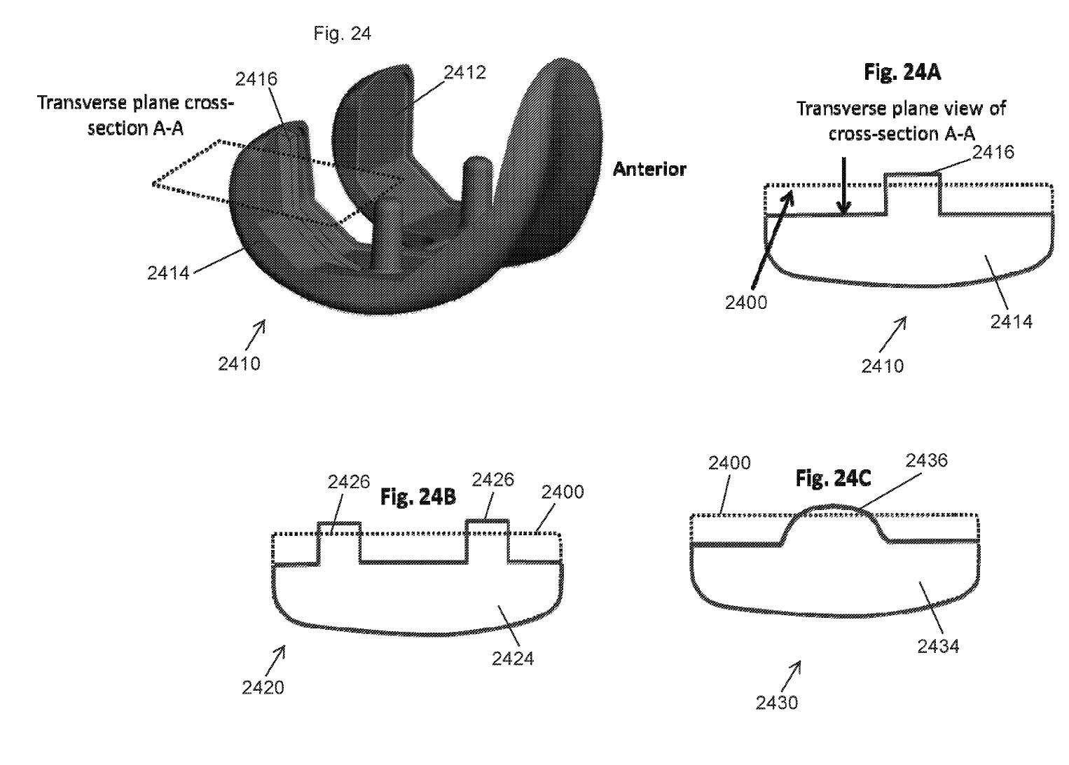

Reduction in thickness of the femoral condyles may reduce strength of the condyle. To address this, in further embodiments of the invention, reinforcing structures such as rectangular fins or semi-cylindrical fins, may be added to the inner surface of one or both of the femoral condyles interfacing with the femoral bone (see FIGS. 24-24C). In FIGS. 24 and 24A, an example femoral component 2410 of the present invention includes a lateral condyle 2412 and a medial condyle 2414 including a rectangular fin 2416 that extends away from the inner surface of the medial condyle 2414. In FIG. 24A, there is a comparison of a conventional lateral condyle having straight inner surface 2400 (in dashed lines) of the femoral condyle and a femoral component 2410 of the present invention having a medial condyle 2414 including a central rectangular fin 2416. In FIG. 24B, there is a comparison of a conventional lateral condyle having straight inner surface 2400 (in dashed lines) of the femoral condyle and a femoral component 2420 of the present invention having a medial condyle 2424 including a pair of spaced apart rectangular fins 2426. In FIG. 24C, there is a comparison of a conventional lateral condyle having straight inner surface 2400 (in dashed lines) of the femoral condyle and a femoral component 2430 of the present invention having a medial condyle 2434 including a central semi-cylindrical fin 2436.

The insertion of the native PCL on the femoral side lies along the mesial side of the medial femoral condyle 2514 (see FIGS. 25A and 25B). The PCL insertion in the average knee has a width wPCL of about 26 mm, a height hACL of about 16 mm. The posterior edge of the PCL insertion is approximately 7 mm from posterior femoral condyle bone surface, approximately 7 mm from distal femoral condyle bone surface, and approximately 5 mm from posterodistal femoral condyle bone surface (see FIGS. 25A and 25B). In a conventional femoral component, the condyle thickness of the implant is greater than or equal to about 8 mm. Thus, removal of corresponding bone from the posterior condyle of the femur to accommodate the femoral implant can result in removal of significant proportion of the PCL insertions and PCL fibers at/near the insertion, thereby weakening the ligament.

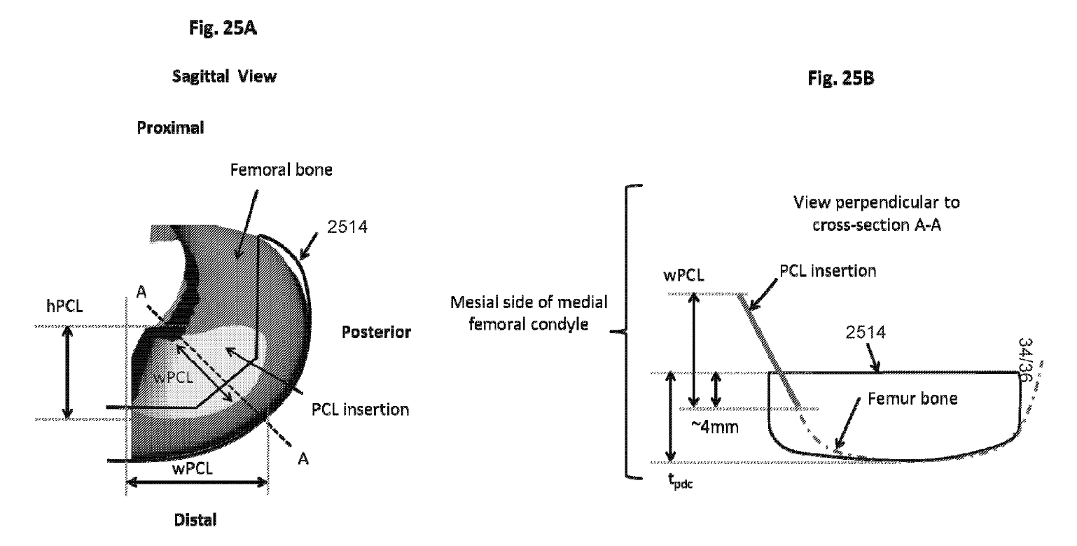

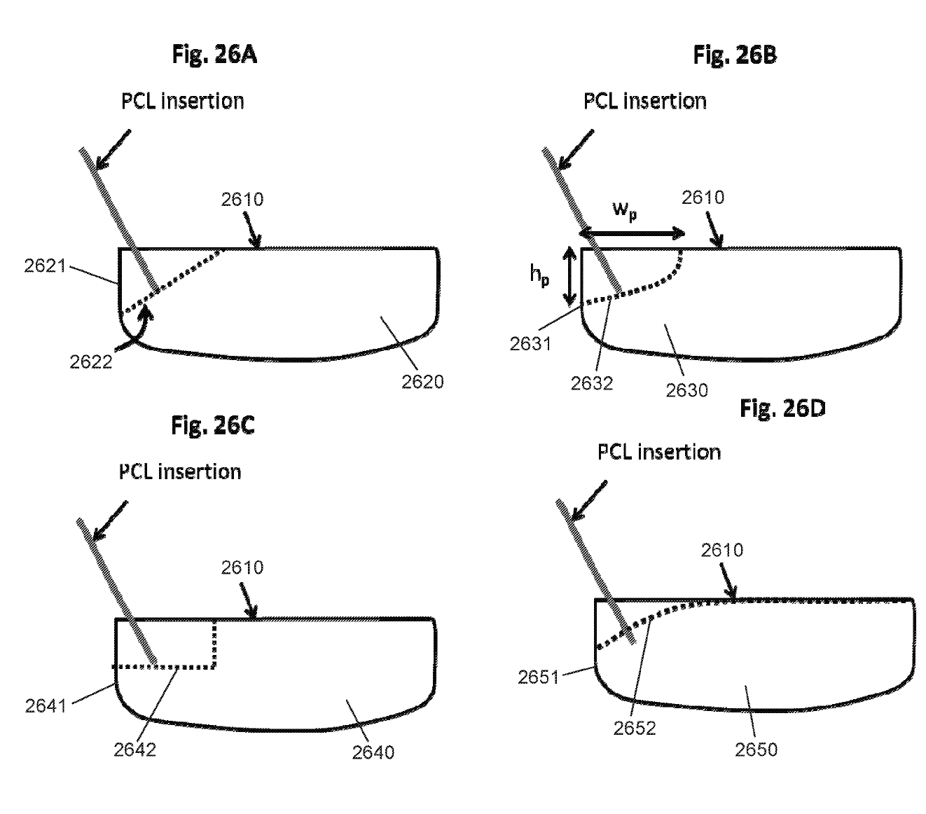

To address this, in one set of embodiments, the geometry of the femoral component is modified to remove material along a mesial edge of the medial condyle (see FIGS. 26A-26D). In FIG. 26A, there is comparison of a conventional medial condyle 2610 and a medial condyle 2620 of the present invention in which a removal of material can be achieved through a gradual chamfer surface region 2622 at the mesial edge 2621. In FIG. 26B, there is comparison of a conventional medial condyle 2610 and a medial condyle 2630 of the present invention in which a removal of material can be achieved through a smooth concave curvilinear arcuate profile surface region 2632 at the mesial edge 2631. In FIG. 26C, there is comparison of a conventional medial condyle 2610 and a medial condyle 2640 of the present invention in which a removal of material can be achieved through a sharp concave transition surface region 2642 at the mesial edge 2641. In FIG. 26D, there is comparison of a conventional medial condyle 2610 and a medial condyle 2650 of the present invention in which a removal of material can be achieved through a sharp convex transition surface region 2652 at the mesial edge 2651. In preferred embodiments, the parameters wₚ and hₚ have a value of about 4 mm, but can range from 0.5 to 7 mm, 2 to 4 mm, etc. The parameters wₚ and hₚ can be measured using the methodology described above for measuring parameters wₐ and hₐ.

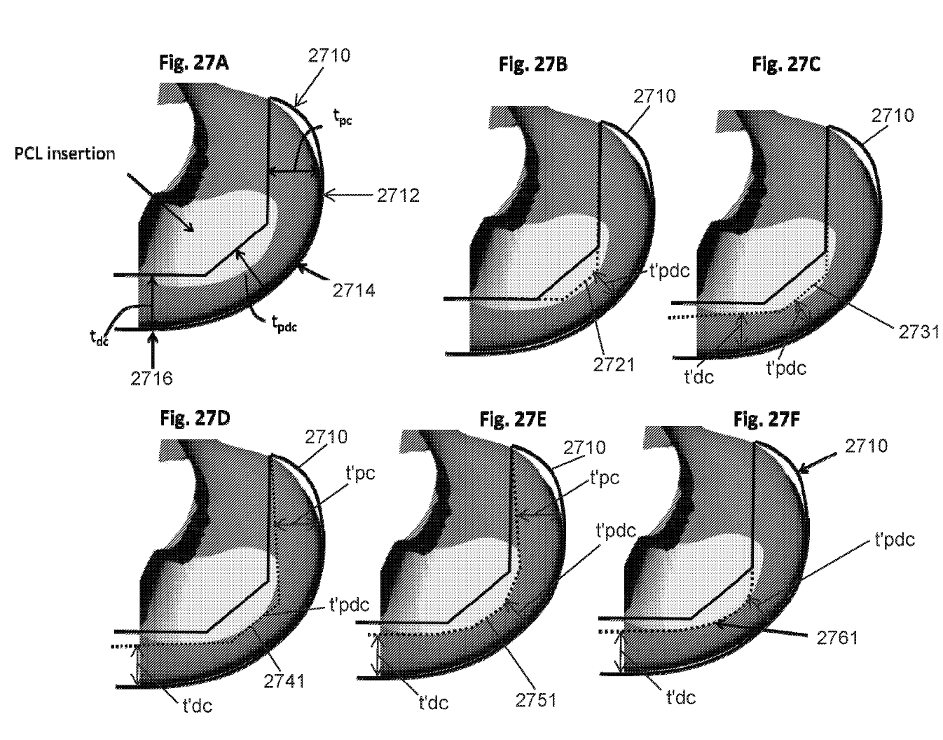

In a conventional femoral component, the posterior, distal and posterodistal condyle thickness (tₚ, t_{dc} and t_{pdc}) are generally equal and about 8 mm or greater (range 8 to 12 mm). In some embodiments of the invention, the thickness of the femoral condyle may be reduced, resulting in modification of the sagittal plane geometry of the inner surface of the femoral implant interfacing with the femoral bone. In some embodiments (FIGS. 27B-27F), the posterodistal condyle thickness (t′pdc) may be reduced to about 5 mm (range 1 mm to 7.5 mm), while the posterior (t′pc) and distal condyle thickness (t′dc) are greater and about 8 mm (range 8 mm to 15 mm). In other embodiments the posterodistal condyle thickness (t′_{pdc}) may be about 5 mm (range 1 mm to 7.5 mm), the distal condyle thickness (t′_{dc}) may be about 6.5 mm (range 1 mm to 7.5 mm), and the posterior condyle thickness (t′_{pc}) may be about 8 mm or greater. In other embodiments, the posterodistal condyle thickness (t′_{pdc}) may be about 5 mm (range 1 mm to 7.5 mm), while the posterior and distal condyle thickness (t′_{pc}, t′_{dc}) may be about 6.5 mm (range 1 mm to 7.5 mm). In the above embodiments, the sagittal geometry of the inner surface of the posterior, posterodistal and distal condyle interfacing with the femoral bone may be composed of one of more straight lines as in embodiments of FIGS. 27A-27E, or may be arcuate as in embodiment of FIG. 27F.

Specifically, FIG. 27A shows a conventional medial condyle 2710 including a posterior condyle 2712, a posterodistal condyle 2714 and a distal condyle 2716 having a posterior condyle thickness (t_{pc}), a posterodistal condyle thickness (t_{pdc}), and distal condyle thickness (t_{dc}), respectively. In FIG. 27B, there is a comparison of the conventional medial condyle 2710 and a medial condyle of the present invention in which an inner surface 2721 (shown in dashed lines) creates reduced posterodistal condyle thickness (t′pdc). In FIG. 27C, there is a comparison of the conventional medial condyle 2710 and a medial condyle of the present invention in which an inner surface 2731 (shown in dashed lines) creates reduced posterodistal condyle thickness (t′pdc) and reduced distal condyle thickness (t′_{dc}). In FIG. 27D, there is a comparison of the conventional medial condyle 2710 and a medial condyle of the present invention in which an inner surface 2741 (shown in dashed lines) creates reduced posterior condyle thickness (t′_{pc}) and reduced posterodistal condyle thickness (t′_{pdc}) and reduced distal condyle thickness (t′_{dc}). In FIG. 27E, there is a comparison of the conventional medial condyle 2710 and a medial condyle of the present invention in which an inner surface 2751 (shown in dashed lines) creates reduced posterior condyle thickness (t′_{pc}) and reduced posterodistal condyle thickness (t′_{pdc}) and reduced distal condyle thickness (t′_{dc}). In FIG. 27F, there is a comparison of the conventional medial condyle 2710 and a medial condyle of the present invention in which an inner surface 2761 (shown in dashed lines) creates reduced posterodistal condyle thickness (t′_{pdc}) and reduced distal condyle thickness (t′_{dc}).

Reduction in thickness of the femoral medial condyles may reduce strength of the condyle. To address this, in further embodiments of the invention, the reinforcing structures such as rectangular or cylindrical fins may be added to the inner surface of the femoral medial condyles interfacing with the femoral bone as in the embodiments of the invention shown in FIGS. 24-24C.

### Prosthesis Materials and Construction:

The prosthetic components can be constructed in various sizes to fit a range of typical patients, or the components can be custom-designed for a specific patient based on data provided by a surgeon, e.g., after physical and radiography examination of the specific patient. The implants described herein can be constructed in various manners and can be made from one or more materials. Implant components (e.g., tibial insert, tibial baseplate, femoral component, tibial cutting block, instrument handle) can be machined, cast, forged, molded, or otherwise constructed out of a medical grade, physiologically acceptable material such as a cobalt chromium alloy, a titanium alloy, stainless steel, ceramic, etc. Other examples of materials for the implants include polyolefins, polyethylene, ultra-high molecular weight polyethylene, medium-density polyethylene, high-density polyethylene, medium-density polyethylene, highly cross-linked ultra-high molecular weight polyethylene (UHMWPE), etc. Exemplary embodiments of UHMWPE prosthesis materials and manufacturing processes are described in U.S. Pat. No. 5,879,400 filed Feb. 13, 1996 entitled “Melt-Irradiated Ultra High Molecular Weight Polyethylene Prosthetic Devices”; U.S. Patent Application Publication No. 2009/0105364 filed Dec. 12, 2008, entitled “Radiation And Melt Treated Ultra High Molecular Weight Polyethylene Prosthetic Devices”; U.S. Pat. No. 7,906,064 filed Nov. 29, 2006 entitled “Methods For Making Oxidation Resistant Polymeric Material”; U.S. Pat. No. 8,293,811 filed Apr. 5, 2010 entitled “Methods For Making Oxidation-Resistant Cross-Linked Polymeric Materials”; U.S. Pat. No. 7,166,650 filed Jan. 7, 2005 entitled “High Modulus Crosslinked Polyethylene With Reduced Residual Free Radical Concentration Prepared Below The Melt”; and U.S. Patent Application Publication No. 2008/0215142 filed Mar. 3, 2008 entitled “Cross-Linking Of Antioxidant-Containing Polymers”, which are hereby incorporated by reference in their entireties.

The devices disclosed herein can be designed to be disposed of after a single use, or they can be designed to be used multiple times. In either case, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to or during a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

One skilled in the art will appreciate further features and advantages of the invention based on the above-described non-limiting embodiments. Accordingly, the invention is not to be limited by what has been particularly shown and described, except as indicated by the appended claims.

All publications and references cited herein are expressly incorporated herein by reference in their entirety.

1. An orthopedic implant comprising:
a femoral implant including one or both of a medial condyle and a lateral condyle,
wherein at least one of the medial condyle and the lateral condyle has a surface region joining a mesial edge and a femur-facing inner surface of the medial condyle or the lateral condyle, and the surface region, when viewed in a plane transversely extending from an outer articular surface to the inner surface of the medial condyle or the lateral condyle, has a concave, convex, or chamfered geometry.

2. The orthopedic implant of claim 1 wherein:
the orthopedic implant is a unicompartmental implant comprising the femoral implant including the medial condyle, and
a surface region joining a mesial edge and a femur-facing inner surface of the medial condyle, when viewed in a plane transversely extending from an outer articular surface to the inner surface of the medial condyle has a concave, convex, or chamfered geometry.

3. The orthopedic implant of claim 1 wherein:
the orthopedic implant is a unicompartmental implant comprising the femoral implant including the lateral condyle, and
a surface region joining a mesial edge and a femur-facing inner surface of the lateral condyle, when viewed in a plane transversely extending from an outer articular surface to the inner surface of the lateral condyle has a concave, convex, or chamfered geometry.

4. The orthopedic implant of claim 1 wherein:
the orthopedic implant is a bi-unicompartmental implant configured to replace both medial and lateral compartments of a native tibia, the bi-unicompartmental implant comprising the femoral implant including the medial condyle and the lateral condyle, and
a surface region joining a mesial edge and a femur-facing inner surface of the medial condyle, when viewed in a plane transversely extending from an outer articular surface to the inner surface of the medial condyle has a concave, convex, or chamfered geometry.

5. The orthopedic implant of claim 1 wherein:
the orthopedic implant is a bi-unicompartmental implant configured to replace both medial and lateral compartments of a native tibia, the bi-unicompartmental implant comprising the femoral implant including the medial condyle and the lateral condyle, and
a surface region joining a mesial edge and a femur-facing inner surface of the lateral condyle, when viewed in a plane transversely extending from an outer articular surface to the inner surface of the lateral condyle has a concave, convex, or chamfered geometry.

6. The orthopedic implant of claim 1 wherein:
the orthopedic implant is a bi-unicompartmental implant configured to replace both medial and lateral compartments of a native tibia, the bi-unicompartmental implant comprising the femoral implant including the medial condyle and the lateral condyle, and
a surface region joining a mesial edge and a femur-facing inner surface of the medial condyle, when viewed in a plane transversely extending from an outer articular surface to the inner surface of the medial condyle has a concave, convex, or chamfered geometry, and
a second surface region joining a mesial edge and a femur-facing inner surface of the lateral condyle, when viewed in a plane transversely extending from an outer articular surface to the inner surface of the lateral condyle has a concave, convex, or chamfered geometry.

7. The orthopedic implant of claim 1 wherein:
the orthopedic implant is a bi-cruciate implant configured to replace both medial and lateral compartments of a native tibia, the bi-cruciate implant comprising the femoral implant including the medial condyle and the lateral condyle, and
a surface region joining a mesial edge and a femur-facing inner surface of the medial condyle, when viewed in a plane transversely extending from an outer articular surface to the inner surface of the medial condyle has a concave, convex, or chamfered geometry.

8. The orthopedic implant of claim 1 wherein:
the orthopedic implant is a bi-cruciate implant configured to replace both medial and lateral compartments of a native tibia, the bi-cruciate implant comprising the femoral implant including the medial condyle and the lateral condyle, and
a surface region joining a mesial edge and a femur-facing inner surface of the lateral condyle, when viewed in a plane transversely extending from an outer articular surface to the inner surface of the lateral condyle has a concave, convex, or chamfered geometry.

9. The orthopedic implant of claim 1 wherein:
the orthopedic implant is a bi-cruciate implant configured to replace both medial and lateral compartments of a native tibia, the bi-cruciate implant comprising the femoral implant including the medial condyle and the lateral condyle, and
a surface region joining a mesial edge and a femur-facing inner surface of the medial condyle, when viewed in a plane transversely extending from an outer articular surface to the inner surface of the medial condyle has a concave, convex, or chamfered geometry, and
a second surface region joining a mesial edge and a femur-facing inner surface of the lateral condyle, when viewed in a plane transversely extending from an outer articular surface to the inner surface of the lateral condyle has a concave, convex, or chamfered geometry.

10. The orthopedic implant of claim 1 wherein:
the surface region, when viewed in the plane, has a straight line profile.

11. The orthopedic implant of claim 1 wherein:
the surface region, when viewed in the plane, has a concave curvilinear profile.

12. The orthopedic implant of claim 1 wherein:
the surface region, when viewed in the plane, has a convex curvilinear profile.

13. The orthopedic implant of claim 1 wherein:
the surface region, when viewed in the plane, has a concave profile comprising a plurality of connected line segments.

14. The orthopedic implant of claim 1 wherein:
a distance, measured perpendicularly from the inner surface to a normal line to a junction of the surface region and the mesial edge, is in a range of range 0.5 to 7 millimeters.

15. The orthopedic implant of claim 1 wherein:
a distance, measured perpendicularly from the mesial edge to a normal line to a junction of the surface region and the inner surface, is in a range of range 0.5 to 7 millimeters.

16. An orthopedic implant comprising:
a femoral implant including one or both of a medial condyle and a lateral condyle,
wherein at least one of the medial condyle and the lateral condyle is configured such that a posterior thickness and/or a posterodistal condyle thickness of the medial condyle or the lateral condyle is less than a distal condyle thickness of the medial condyle or the lateral condyle.

17. The orthopedic implant of claim 16 wherein:
the orthopedic implant is a unicompartmental implant comprising the femoral implant including the medial condyle, and
the medial condyle is configured such that a posterior thickness and/or a posterodistal condyle thickness of the medial condyle is less than a distal condyle thickness of the medial condyle.

18. The orthopedic implant of claim 16 wherein:
the orthopedic implant is a unicompartmental implant comprising the femoral implant including the lateral condyle, and
the lateral condyle is configured such that a posterior thickness and/or a posterodistal condyle thickness of the lateral condyle is less than a distal condyle thickness of the lateral condyle.

19. The orthopedic implant of claim 16 wherein:
the orthopedic implant is a bi-unicompartmental implant configured to replace both medial and lateral compartments of a native tibia, the bi-unicompartmental implant comprising the femoral implant including the medial condyle and the lateral condyle, and
the medial condyle is configured such that a posterior thickness and/or a posterodistal condyle thickness of the medial condyle is less than a distal condyle thickness of the medial condyle.

20. The orthopedic implant of claim 16 wherein:
the orthopedic implant is a bi-unicompartmental implant configured to replace both medial and lateral compartments of a native tibia, the bi-unicompartmental implant comprising the femoral implant including the medial condyle and the lateral condyle, and
the lateral condyle is configured such that a posterior thickness and/or a posterodistal condyle thickness of the lateral condyle is less than a distal condyle thickness of the lateral condyle.

21-107. (canceled)
